SEQUENCE LISTING (2 sequences)

SEQ ID NO: 1 - DNA, 26 bp, Artificial Sequence
  Description of Artificial Sequence Synthetic primer
gtagaagcgaattcaacaacatcgac

SEQ ID NO: 2 - DNA, 20 bp, Artificial Sequence
  Description of Artificial Sequence Synthetic primer
ggttcgaattcatgggttcc

(19) United States
(12) Patent Application Publication
(10) Pub. No.: US 2016/0251614 A1
(43) Pub. Date: Sep. 1, 2016

(54) CELL CULTURE MEDIUM AND BIOPROCESS OPTIMIZATION

(71) Applicant: UNIVERSITY OF NOTRE DAME DU LAC, Notre Dame, IN (US)

(72) Inventors: Jeremiah ZARTMAN, Granger, IN (US); Miranda BURNETTE, South Bend, IN (US); Jun LI, Granger, IN (US); Teresa BRITO-ROBINSON, South Bend, IN (US)

(73) Assignee: University of Notre Dame du Lac, Notre Dame, IN (US)

(21) Appl. No.: 15/033,531

(22) PCT Filed: Nov. 3, 2014
(86) PCT No.: PCT/US14/63725

§ 371 Date: Apr. 29, 2016

Related U.S. Application Data
(60) Provisional application No. 61/898,852, filed on Nov. 1, 2013.
(60) Provisional application No. 62/027,916, filed on Jul. 23, 2014.

(51) Int. Cl. C12N 5/07; G06F 19/28; G06F 19/18; G01N 33/50

(52) U.S. Cl. CPC C12N 5/0601; G01N 33/5044; G06F 19/28; G06F 19/18; C12N 2501/33; C12N 2500/30; C12N 2500/38; C12N 2500/32; C12N 2501/727; C12N 2500/46; C12N 2500/14; C12N 2500/24; C12N 2500/22; C12N 2500/20; C12N 2500/36; C12N 2500/42

(57) ABSTRACT

The invention provides a chemically defined cell culture media and methods of using the media. The invention also provides an inverse screen to identify small molecules and synergies stimulating proliferation in a chemically defined medium. In this chemical-genetics approach, a compound-protein interaction data-base is used to systematically score genetic targets on a screen-wide scale to extract further information about cell growth. Validated factors were investigated for their ability to maintain cell growth over multiple passages in the chemically defined medium (CDM). Polyamines were identified as important components that enables the CDM to support the long-term maintenance of C1.8 cells and Kc cells (such as Kc167 cells). Our cumulative target scoring approach improves on traditional chemical-genetics methods and is extensible to biological processes in other species.

### RELATED APPLICATIONS

This application claims priority under 35 U.S.C. §119(e) to U.S. Provisional Patent Application No. 61/898,852, filed Nov. 1, 2013 and 62/027,916, filed Jul. 23, 2014, which applications are incorporated herein by reference.

### BACKGROUND OF THE INVENTION

Chemical genetics has recently emerged as a complimentary method to traditional genetics where the central theme is the use of small molecules for studying biological systems. One especially promising form of chemical genetics is inverse drug screening, where known bioactive compounds are screened for phenotypes. This inverse approach is analogous to RNA interference (RNAi) screens in that the compounds all have known or putative targets, and thus compound “hits” provide insights into the biological pathways involved in the process of interest. Some advantages of the small molecule approach are speed, reversibility, wide applicability across species, and efficiency (one compound can probe multiple putative targets). These screens have been harnessed to investigate multiple aspects of biology including mitosis, pigmentation, development, insulin signaling, and wound healing. In particular, Drosophila melanogaster has been used for whole organism scale small molecule screens to study various biological processes.

Drosophila is a versatile model system used to understand the development and physiology of multiple tissue types. Traditionally, the unsurpassed genetic and molecular tools available for in vivo studies has relegated the development of in vitro tools to a secondary role. However, for increased throughput and ease, Drosophila cell and organ culture is becoming more widespread, especially in genetic and chemical screens (FIG. 14A-B). Still, Drosophila cell culture tools are relatively undeveloped, limiting the utility of Drosophila cell culture as a model system. In particular, there is currently no acceptable chemically defined medium (CDM) available for the culture of Drosophila cell lines; current media either include undefined extracts (e.g., yeast extract) or require supplementation with undefined and highly variable serum such as fetal bovine serum (FBS) or fly extract (FEX). These undefined supplements limit the control and reproducibility of cell culture experiments, and due to their complex nature hinder proteomic analyses.

The most recent attempts to rationally design chemically defined media for Drosophila cell culture were made over 30 years ago, before the development and spread of high-throughput screening techniques (Br. J. Pharmacol., 2007, 152, 53-61). Wyss' systematic attempt was based on the requirements of two embryonic cell lines, Kc and Ca (Wyss, Exp. Cell Res., 1982, 139, 297-307). The resulting formulation, ZO media, could support Kc cell growth upon inclusion of fly extract, and was also used to create an epithelial-like cell line from Chironomus tentans upon supplementation with FBS, yeast extract, and insulin (Wyss, Exp. Cell Res., 1982, 139, 309-319).

Applied to culture media design, small molecule screens have previously been used to identify media supplements enabling self-renewal of embryonic stem cells and to find inducers of B-cell expansion (Wang et al., Proc. Natl. Acad. Sci. U.S.A., 2009, 106, 1427-1432). Similarly, a compound cocktail was optimized to support long-term growth of human embryonic stem cells using five predefined candidate molecules associated with known pathways (Tsutsui et al., Nat. Commun., 2011, 2, 167). However, to our knowledge no systematic attempt has been made to both identify growth promoters and optimize Drosophila culture media in a high-throughput fashion.

Accordingly, there is a need for new chemically defined media for the growth and maintenance of Drosophila cell lines, particularly long-term growth and proliferation. There is also a need for methods for screening Drosophila cells to enable the identification of compounds that stimulate cell proliferation, and the identification of genetic targets and biological pathways important for cell growth.

### SUMMARY

The invention provides an aqueous chemically defined media for supporting long-term growth of cells such as insect cell lines, for example, Drosophila cell lines. The media includes ZO media or ZW media, which media is supplemented with insulin, a disaccharide, ascorbic acid, and at least one of glutamine and glutamate. The pH of the media can be about 6 to about 8. The media can be a minimal media and exclude non-essential components such as fetal bovine serum, fly extract, yeast extract, bactopeptone, or a combination thereof. Thus, the media can be serum-free and completely chemically defined.

In one embodiment, the media comprises at least about 94% water by weight. Typically the media is about 95% to about 99% water by weight.

In various embodiments, insulin is present at a concentration of at least about 1 ng μg/mL. In some embodiments, the insulin is present at a concentration of about 1 ng μg/mL to about 50 μg/mL, typically about 1 ng/mL-10 μg/mL, about 2.5 μg/mL to about 10 μg/mL, or about 5 μg/mL.

In various embodiments, the disaccharide can be present at a concentration of at least about 10 mM. In some embodiments, the disaccharide is present at a concentration of about 10 mM to about 40 mM, about 20 mM to about 30 mM, or about 25 mM. In some embodiments, the disaccharide can be a nonreducing disaccharide. In one embodiment, the disaccharide is trehalose. In other embodiments, the disaccharide can be maltose or fructose.

In various embodiments, the ascorbic acid is present at a concentration of at least about 40 ng/mL. In some embodiments, the ascorbic acid is present at a concentration of about 50 mM to about 250 mM, about 50 mM to about 150 mM, about 60 mM to about 100 mM, or about 80 mM. In one embodiment, the ascorbic acid is in the form of L-ascorbic acid. In some embodiments, the ascorbic acid is in the form of an ascorbic acid 2-phosphate salt, such as the sesquimagnesium salt hydrate, although a variety of ascorbic acid esters and salts may be employed.

In various embodiments, at least one of glutamine and glutamate is present at a concentration of at least about 5 μg/mL. The glutamine or the glutamate can be in the form of a mono-, di-, or tri-peptide. One advantageous form of glutamine is the dipeptide alanyl-glutamine, although other di- and tri-peptides comprising glutamine or glutamate can be used.

In various embodiments, the pH of the media is greater than 6. In various embodiments, the pH of the media is less than 8. In some embodiments, the pH of the media is about 6 to about 8, about 6.5 to about 7.3, about 6.6 to about 7.2, about 6.7 to about 6.8, or about 6.75.

The media can also include a polyamine compound. The polyamine compound can be present in the media at a concentration of at least about 0.1 μM. In various embodiments, the concentration of the polyamine compound can be at least about 0.25 μM or at least about 0.5 μM. In some embodiments, the concentration of the polyamine compound can be about 0.25 μM to about 40 μM, 0.5 μM to about 20 μM, 1 μM to about 20 μM, or about 10 μM. The polyamine can be a diamine, a triamine, a tetraamine, an oligoamine, or a polyamine. In one embodiment, the polyamine is spermidine, spermine, putrescine, or a combination thereof. In one specific embodiment, the polyamine is spermidine. In another specific embodiment, the polyamine is spermine. In yet another specific embodiment, the polyamine is putrescine.

The media can include a BTK inhibitor. In some embodiments, the BTK inhibitor is terreic acid or LFM-A13 (α-cyano-β-hydroxy-β-methyl-N-(2,5-dibromophenyl)propen-amide).

In one embodiment, the media includes insulin at a concentration of about 3-10 μg/mL, a disaccharide at a concentration of about 20-30 mM, L-alanyl-L-glutamine at a concentration of about 10-20 μM, and L-ascorbic acid 2-phosphate sesquimagnesium salt hydrate at a concentration of about 50-150 nM. The pH of the media can be about 6 to about 8, or about 6.7 to about 6.8. The media can include a polyamine at a concentration of about 0.5 μM to about 20 μM. In some specific embodiments, the polyamine is spermidine.

The invention also provides an aqueous serum-free chemically defined media for supporting long-term growth of Drosophila cell lines, the media consisting essentially of ZO media supplemented with insulin at a concentration of about 1 ng/mL-10 μg/mL, a disaccharide at a concentration of about 20-30 mM, L-alanyl-L-glutamine at a concentration of about 1-20 μM, L-ascorbic acid 2-phosphate sesquimagnesium salt hydrate at a concentration of about 40-150 nM, and at least one of glutamine and glutamate at a concentration of about 1 μM to about 20 μM, wherein the pH of the media is about 6 to about 8. In other embodiments, the media consists of the aforementioned components.

The invention further provides a method for screening small molecule compounds. The method can include a) combining Drosophila cells, one or more test compounds, and a chemically defined cell growth media such as a Drosophila cell media. The media can be a minimal media such as a serum-free media and/or a completely chemically defined media, for example, ZO media or ZW media. The cell media can be media described herein, such as an aqueous chemically defined media comprising ZO media or ZW media supplemented with insulin, a disaccharide, L-ascorbic acid 2-phosphate, and at least one of glutamine and glutamate, wherein the pH of the media is about 6 to about 8. The method can also include: b) analyzing the cells after a period of time to score the screen for increases or decreases in the number of cells that are in the presence of the one or more test compounds to obtain compound proliferation z-scores (zᵢ). The analysis can include assays for quantifying fluorescence and/or cell number, such as with the CyQUANT® Direct Proliferation Assay, or by cell signaling or use a transfected cell line that expresses a reporter of cell signaling (see FIG. 23, which shows GCaMP6, a sensor of intracellular calcium signaling). The method can also include c) identifying test compounds that increase the number of Drosophila cells in the media and test compounds that modify the number of Drosophila cells in the media, change the morphology of the cells, or affect the amount of cell signaling as measured by reporter genes or chemical dyes (and scoring compounds for their effect on cell number and selecting hit compounds that induce a certain effect size), thereby identifying compounds of interest (“hit compounds”). The method can further include d) querying the Search Tool for Interactions of Chemicals (STITCH) database for identified test compounds to determine test compound-protein interaction scores (sᵢ), thereby identifying proteins that interact with the test compounds, or test compounds that interact with certain proteins; and e) optionally identifying species homologs and percent identity matches (qᵢ) for orthologous Drosophila proteins to identified mouse or human protein-drug interactions (e.g., to translate putative human/drug (test compound) or mouse/drug (test compound) interactions to Drosophila/drug interactions); thereby translating screen wide compound proliferation data into biologically relevant protein data, and optionally identifying proteins that interact with the test compounds, or test compounds that interact with certain proteins. The significance of protein targets (p-values) can be determined from the protein score, Σ(sᵢ·qᵢ·zᵢ²), which follows a chi-squared distribution with degrees of freedom n.

The method can be a high-throughput screen. For example, in some embodiments, the method is carried out in parallel with one or more negative controls and/or at least about 10 different test compounds, optionally in a 96-well format. The method can be repeated one or more times including test compounds identified in the previous iteration (e.g., step “c)”) as increasing cell number, or that have a test proliferation score (zᵢ) of greater than a specific value of interest in background to specifically search for identify putative interactions.

The methods described herein can also include generating rationally designed small molecule libraries based on known pathways/targets of interest. The methods can also include generating drug-pathway interaction scores (dₓ) from cumulative score of drug-protein interactions within the pathway (s_{i,x}) (e.g., from STITCH) and penalized by interactions with proteins in other pathways (s_{i,y}), thereby scoring a list of test compounds for their activity and specificity to given pathways of interest (e.g., cumulatively scoring a biological pathway relevancy for a given process). The method can further include selecting compounds with the highest biological pathway relevancy to generate a library with highest pathway specificity, coverage, efficiency, and nonredundancy of the pathway of interest. The library can be screened using multifactorial design of experiments methodology to specifically probe for pathway interactions. In various embodiments, the chemically defined media comprises a polyamine.

The invention additionally provides a method for translating compound phenotypic screen scores to biological target scores and enriched ontology terms relevant to a process of interest comprising screening a library of compounds and insect cells in a media described herein and using the Search Tool for Interactions of Chemicals (STITCH) compound-protein interaction database to translate the compound phenotypic screen scores to biological target scores and enriched ontology terms relevant to the process of interest. The invention also provides methods for rationally designing compound libraries based on cataloged drug-protein interactions in STITCH and other databases. The methods can include generating a list of proteins of interest (or protein products of the entire proteome), querying the STITCH database to identify compounds targeting those proteins of interest and the associated interaction score, and applying an optimization strategy to generate a small library list (e.g., a library of less than a desired threshold number) that probes the proteins or pathways of interest with a minimum interaction score. The invention yet further provides a high-throughput inverse drug-screening platform to identify novel compounds and genetic targets important for proliferation of Drosophila cells. By systematically identifying and scoring protein targets of the screened compounds, genes and pathways, in addition to compounds important for growth, can be identified. Cumulative small molecule scores can be translated to gene target scores using a chemical-protein interaction database to elucidate targets with small effects (e.g. small effects from multiple compounds with same target) (see FIG. 14E). The invention also provides a method to develop rationally designed compound libraries targeting a high level of genome coverage or target-of-interest coverage using a minimal size library based on cataloged protein-compound interactions.

The invention thus provides novel chemically defined media and methods of preparing the media. The invention also provides chemically defined media that are useful as cell culture media for a variety of insect species. The media can also be used for culturing cells for screening, such as high-throughput screening, for improving viable cell density, for reducing cell doubling time, and/or for transfection and expression of heterologous recombinant proteins. The library generation methods described herein can be applied to other processes of interest other than and in addition to preparing chemically defined media for Drosophila cells.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Effect of basal media on C1.8 proliferation: (A) growth ratios, and (B) visualization of cells. Asterisks indicate significant differences (p<0.05).

FIG. 2. Representative positive and negative control wells from primary screen of C1.8 and S2 cells in ZO Fortified.

FIG. 3. Compound z-scores for primary screen in ZO Fortified on (A) C1.8 cells, (B) S2 CELLS. Bolded z-scores denote hit compounds, asterisks denote repeat hit compounds. (C) Positive hit compounds from primary screen on C1.8 cells in ZO Fortified.

FIG. 4. Venn diagram of positive hits from primary screen on C1.8 and S2 cells in ZO Fortified.

FIG. 5. (A) Repeated negative hits (22) from primary screen on C1.8 and S2 cells in ZO Fortified; (B) Negative hits from primary screen on C1.8 cells in ZO Fortified; (C) Negative hits from primary screen on S2 cells in ZO Fortified.

FIG. 6. Top positive hits from primary screen on (A) C1.8 cells (B) S2 cells in ZO Fortified.

FIG. 7. Representative positive and negative control wells from primary screen of C1.8 and S2 cells in ZB Media.

FIG. 8. Compound z-scores for secondary screen in ZB Media on (A) C1.8 cells, (B) S2 cells.

FIG. 9. Negative hit compounds from secondary screen on C1.8 cells in ZB Media. Bolded z-scores denote hit compounds, asterisks denote repeat hit compounds.

FIG. 10. Negative hit compounds from secondary screen on S2 cells in ZB Media. Bolded z-scores denote hit compounds, asterisks denote repeat hit compounds.

FIG. 11. Venn diagram of positive hits from secondary screen on C1.8 and S2 cells in ZB Media.

FIG. 12. Venn diagram of negative hits from secondary screen on C1.8 and S2 cells in ZB Media.

FIG. 13. Strongest positive and negatives hits primary screen on (A) C1.8 cells (B) S2 cells in ZB Media.

FIG. 14. (A): Advantages and limitations of in vivo and in vitro experimentation. The precise control offered by in vitro culture is abrogated by the required medium supplementation with undefined extracts. (B): Comparison of mammalian versus Drosophila cell culture. (C): Comparison of C1.8 growth kinetics in complete serum containing media, ZO media unsupplemented, and “ZO Fortified.” ZO Fortified supports initial attachment and proliferation of C1.8 cells whereas ZO unsupplemented does not. Single and double asterisks denote p<0.05 and p<0.01, respectively, for two-tailed t-test for unmatched pairs. (D): Screening pipeline. Small molecule libraries are screened on two cell lines for their ability to promote proliferation in a minimal, serum-free medium. Putative hit compounds are then compared between cell lines to minimize false positives, and analysis on putative targets is conducted. The screen can be iterated upon to identify compound synergies by incorporating hit compounds into the background media and rescreening. (E): Target scoring pipeline. Compound-protein interaction scores (sᵢ) are identified from a database and linked to compound z-scores. Non-Drosophila protein targets are “translated” to their Drosophila orthologs retaining their percent identity match score, qᵢ. Drosophila protein target p-values are then calculated by summing for each interaction with a compound its compound-protein interaction score, percent identity match scores, and squared compound z-scores (which follows a chi-squared distribution). This analysis yields a list of significantly targeted proteins for a process of interest. Protein target lists can be also converted to their encoding genes and gene ontology enrichment can be performed. See Example 2.

FIG. 15. (A-B): Z-scores for all screened compounds on (A) C1.8 cells and (B) S2 cells. (C-D): Representative images of hit compounds on C1.8 (C) and S2 (D) cells. (E-F): Venn diagrams for unique positive (E) and negative (F) hit compounds.

FIG. 16. Growth kinetics of Clone 8, Schneider 2, and Kc167 cells in ZO unsupplemented, ZO Fortified, ZB Media, and their respective complete, serum-containing media. Error bars represent standard deviations. (A-C) Changes in averaged fluorescent intensity measurements indicate changes in DNA content from viable cells. Error bars represent standard deviations. (D-F) Fold changes in intensity from day 0 are taken to represent fold changes in cell number. Each of the three cell lines proliferate significantly better in ZB Media (which includes spermidine supplementation) than ZO Fortified by the fifth day. Kc167 cells in particular proliferate at comparable rates to the complete, serum-containing medium by day 5. Error bars represent standard deviations for which propagation of error is accounted. Single and double asterisks denote p<0.05 and p<0.01, respectively, for two-tailed t-test for unmatched pairs. Corresponding p-values can be found in the electronic supplementary information. (G-I) Cells from day five of the same experiment. The apparent low confluency of Kc167 cells (I) is due to a high number of suspension cells that cannot be seen in one plane of view. (J) C1.8 and S2 cells during long term culture in ZO Fortified versus ZB Media; images at the point of discernable difference in cell growth between ZO Fortified and ZB Media. S2 cells passaged into ZO Fortified were unable to reach passage 2 (confluency) whereas those supplemented with spermidine (ZB Media) were able to reach confluency and undergo two population doublings before growth rates stalled (passage 4). C1.8 cells passaged into ZO Fortified became confluent and underwent one population doubling (passage 3) before growth stopped. C1.8 cells cultured in ZB Media are able to proliferate long-term (currently passage 35). C1.8 cells after adaptation (10 passages in ZB Media) can be frozen and thawed successfully.

FIG. 17. (A-B) Proliferation of polyamine-depleted C1.8 cells increases in a dose-dependent manner with spermidine supplementation. Error bars represent standard deviations with for which propagation of error is accounted. Single and double asterisks denote p<0.05 and p<0.01, respectively, for two-tailed t-test for unmatched pairs. (C) Spermidine supplementation (ZB Media) results in increased EdU incorporation in both C1.8 and S2 cells. Error bars represent standard deviations. Single and double asterisks denote p<0.05 and p<0.01, respectively, for two-tailed t-test for unmatched pairs. (D-E) Representative images of EdU (magenta) and DAPI (blue) stained C1.8 (D) and S2 (E) cells in each culture medium tested.

FIG. 18. Spermidine supplementation (ZB Media) results in increases in ERK phosphorylation (dpERK) for C1.8 cells (p<0.012), but not for S2 cells (p>0.75). Although ERK expression does not change significantly with spermidine supplementation for either C1.8 (p>0.55) or S2 (p<0.45) cells, there is a trend of increasing ERK expression across ZO Media (unsupplemented) improvements. Error bars represent standard deviations across three replicates. Single and double asterisks denote p<0.05 and p<0.01, respectively, for two-tailed t-test for unmatched pairs. Corresponding p-values can be found in the electronic supplementary information of Burnette et al. Mol. BioSyst., 2014, 10, 2713-2723, which is incorporated herein by reference.

FIG. 19. REVIGO scatter plots of enriched ontology terms for targets of (A) positive scoring compounds for C1.8 cells, (B) positive scoring compound for S2 cells, (C) negative scoring compounds for C1.8 cells, and (D) negative scoring compounds for S2 cells. Enriched ontology annotations are grouped into terms by semantic similarity and plotted on semantic axes, where similar terms are clustered closely on the plot. Circles representing terms are color-coded according to p-value and sized according to the number of ontology annotations per term.

FIG. 20. Illustration of higher recombinant protein expression in ZB Media compared to Insect-XPRESS™ Insect Cell Medium for S2 cells through four days.

FIG. 21. Illustration a larger picture showing the higher recombinant protein expression in ZB Media compared to Insect-XPRESS™ Insect Cell Medium for S2 cells at Day 4.

FIG. 22. Graph showing that ZB Media has lower background fluorescence than Insect-XPRESS™ Insect Cell Medium for vn-GFP stably transfected S2 cells in either ZB Media or Insect-XPRESS™ Medium quadruplicate (same experiment as shown by FIG. 20). Asterisk denotes p<1E-6 for two tailed t-test for unmatched pairs.

FIG. 23. ZB Media yields comparable transfection efficiency to C1.8 Media for adapted C1.8 cells. C1.8 cells adapted to ZB Media were seeded at 200,000 cell/mL in either C1.8 or ZB Media. After one day, cells were transiently transfected with B-delta2-HA4CaTTGCamp6Fast using TransIT-X2 transfection reagent. Cells were then imaged 48 hours after transfection cells at 10 second intervals for 2 hours.

FIG. 24. Schematics of the insect cell calcium sensor plasmids: A) pAc5-GCaMP6f and B) pΔTub̂att-GCaMP6f.

FIG. 25. High Passage C1.8 cells pΔTub̂att-GCaMP6f Transfection 24 hours post transfection in Spent ZB Media (Evos 20×) (arrows identify fluorescence).

### DETAILED DESCRIPTION

The invention provides a chemically-defined medium for long-term maintenance of insect cells, such as Drosophila cells, by incorporating into the medium a unique combination of compounds that facilitate the long-term proliferation of the cells. Drosophila cell culture is used as a model system with multiple applications including the identification of new therapeutic targets in screens, the study of conserved signal transduction pathway mechanisms, and as an expression system for recombinant proteins. However, the relative lack of in vitro methods for Drosophila cell and tissue culture compared to mammalian cell culture technology limits the current potential of Drosophila research as a biochemical model system for studying human diseases.

To define and characterize the minimal essential requirements for maintaining long-term growth of Drosophila cell lines, we have performed a medium-throughput screen for small molecule compounds that promote cell proliferation in a chemically defined medium. By using an adaptive screening strategy on two cell lines we can identify hit compounds as well as potential synergies between compounds. Validated factors were then investigated for their long term-potential to maintain cell viability and growth over multiple passages in the chemically defined medium in both C1.8 and S2 cells. We found that the polyamines such as spermidine are important, if not critical, for enabling long-term maintenance of C1.8 cells in a chemically defined medium, and that Bruton's tyrosine kinase inhibition is also an important target to stimulate proliferation in this CDM. Iterations of the method enable the identification of an optimal combination of small molecules that can support the maintenance and generation of new Drosophila cell lines, provide advantageous conditions for biochemical studies, and facilitate purification of recombinant proteins, thus increasing the versatility of Drosophila cell lines as both a genetic and biochemical model system.

To characterize the minimal requirements for long-term maintenance of Drosophila cell lines, we also developed an inverse screening strategy to identify small molecules and synergies stimulating proliferation in a chemically defined medium. In this chemical-genetics approach, a compound-protein interaction database is used to systematically score genetic targets on a screen-wide scale to extract further information about cell growth.

In the pilot screen, we focused on two well-characterized cell lines, Clone 8 (C1.8) and Schneider 2 (S2). Validated factors were investigated for their ability to maintain cell growth over multiple passages in the chemically defined media (CDM) described herein. The polyamine spermidine proved to be an important component that enables the CDM to support the long-term maintenance of C1.8 cells. Spermidine supplementation upregulates DNA synthesis for C1.8 and S2 cells and increases MAPK signaling for C1.8 cells. The CDM also supports the long-term growth of Kc167 cells. Our target scoring approach validated the importance of polyamines, with enrichment for multiple polyamine ontologies found for both cell lines. Iterations of the screen can enable the identification of compound combinations optimized for specific applications—maintenance and generation of new cell lines or the production and purification of recombinant proteins—thus increasing the versatility of Drosophila cell culture as both a genetic and biochemical model system. Our cumulative target scoring approach improves on traditional chemical-genetics methods and is extensible to biological processes in other species.

By performing a pilot inverse small molecule screen on Drosophila cells one can identify 1) novel compounds and compound synergies stimulating cell proliferation, 2) genetic targets and biological pathways important for growth, and 3) a combination of compounds sufficient for long-term growth and maintenance of Drosophila cell lines in a chemically defined medium.

### DEFINITIONS

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to bring about a recited effect, such as an amount necessary to form products in a reaction mixture or to promote growth in a cell culture. Determination of an effective amount is typically within the capacity of persons skilled in the art, especially in light of the detailed disclosure provided herein. The term “effective amount” is intended to include an amount of a compound or reagent described herein, or an amount of a combination of compounds or reagents described herein, e.g., that is effective to promote growth in a cell culture. Thus, an “effective amount” generally means an amount that provides the desired effect.

The terms “promote”, “promoting”, and “promotion” with respect to cell growth and/or adhesion refers to the facilitating, increasing, or enhancing the growth, proliferation or progression of a cell or group of cells. The promotion can be greater than about 20%, 40%, 60%, 80%, 90%, 95%, or 99%, for example, compared to the growth or progression that occurs in the absence of the treatment of or contacting by, for example, a media described herein.

The terms “inhibit”, “inhibiting”, and “inhibition” refer to the slowing, halting, or reversing the growth, proliferation or progression of a cell or group of cells. The inhibition can be greater than about 20%/a, 40%, 60%, 80%, 90%, 95%, or 99%, for example, compared to the growth or progression that occurs in the presence of the treatment of or contacting by, for example, a media described herein.

A “chemically defined media” (CDM) refers to a growth medium wherein each component is chemically identifiable and quantifiable and which is serum-free and free of components that can unintentionally vary from batch to batch.

“ZO media” refers to a chemically defined Drosophila culture media developed by Wyss, which replaced yeast extract with trace metals, vitamins, and purines and pyrimidines. ZO media supports Kc cell growth upon inclusion of fly extract. ZO media includes the components below and is prepared as follows.

ZO Medium is prepared at 2× strength, and is made from a base solution supplemented with vitamin, lipid, and minor salt stock solutions, according to the following instructions.

### Overall Instructions

A. Prepare Vitamin Stock Solution (10 mL, 1000×) for ZO Medium

1) Dissolve all components in 10 mL of water

2) Adjust pH to 6.0

3) Aliquot excess and store at −20° C.

B. Prepare Lipid Stock Solution (1 L, 1000×) for ZO Medium

1) Dissolve all components in 1 L of water

2) After extensive stirring, filter out all undissolved material using 0.2 um filter

3) Aliquot excess and store at −20° C.

C. Prepare Minor Salt Solution (10 mL, 1000×) for ZO Medium

1) Dissolve all components in 10 mL of water

2) Aliquot excess and store at −20° C.

D. Prepare Basal ZO Media (1 L, 2× Concentrated)

1) Prepare base ZO Media by dissolving all components (except for calcium chloride) in 300 mL Milli-Q water. Adjust pH to 6.0

2) Add 1 mL of 1000× lipid stock solution

3) Add 1 mL of 1000× vitamin stock solution

4) Add 1 mL of 1000× minor salt solution

5) Adjust pH to 6.6

6) Add calcium chloride dissolved in 50 mL MilliQ water

7) Bring the volume to 500 mL with MilliQ water

8) Filter sterilize with 0.2 um and store for use

### A. Vitamin Stock Solution (at ×1000 10 mL H₂O, Adj. pH to 6.0, Aliquot and Store at −20° C.):

| | Concentration | |
|---|---|---|
| Component | (mg/L) | mg |
| Vitamin B-12 (Cobalamin) | 0.1 | 1 |
| Biotin | 0.1 | 1 |
| Choline Chloride | 20 | 200 |
| Folic Acid | 0.1 | 1 |
| Niacinamide | 0.1 | 1 |
| D-Calcium Pantothenate | 10 | 100 |
| Pyridoxal Hydrochloride (Vitamin B6) | 30 | 300 |
| Riboflavin | 0.01 | 0.1 |
| Thiamine Hydrochloride (Vitamin B1) | 10 | 100 |
| m-Inositol | 20 | 200 |
| Nicotinic Acid (B3) | 10 | 100 |
| Pyridoxine | 0.1 | 1 |

The Vitamin Stock Solution is prepared by dissolving all components in 10 mL of water. Aliquot excess and store at −20° C.

### B. Lipid Stock Solution (at ×1000 in 1000 mL H₂O, Aliquot and Store at −20° C.):

| | Lipid Stock | |
|---|---|---|
| | Concentration | |
| Component | (mg/L) | mg |
| L-a-Lecithin (distearoyl) | 10 | 10 |
| L-a-Lecithin (dimyristoyl) | 10 | 10 |
| L-a-lecithin (dipalmityl) | 5 | 5 |
| sodium-a-glycerophosphate | 160 | 160 |
| calcium phosphoryl choline | 130 | 130 |
| phosphorylethanolamine | 70 | 70 |
| cholestrol | 2 | 2 |
| oleic acid | 50 | 50 |
| linoleic acid | 50 | 50 |

The Lipid Stock Solution is prepared by dissolving all components in 1 L of water. After extensive stirring, filter out all undissolved material using a 0.2 μm filter. Aliquot excess and store at −20° C. In certain embodiments, the media includes each of the nine lipids of the table above, while other embodiments can include one, one or two, one to three, one to four, one to five, one to six, one to seven, or one to eight of any of the lipids recited in the table above.

### C. Minor Salt Stock Solution (at ×1000 in 10 mL 1 mM HCl, Aliquot and Store at −20° C.):

| | Concentration | |
|---|---|---|
| Component | (mg/L) | mg |
| Ferrous Sulfate•7H2O | 0.95 | 9.5 |
| Zinc Sulfate•7H2O | 0.99 | 9.9 |
| CuSO4•5H2O | 0.0016 | 0.016 |
| Manganese Chloride•4H2O | 0.69 | 6.9 |

The Minor Salt Solution is prepared by dissolving all components in 10 mL of water. Aliquot excess and store at −20° C.

### D. ZO Media Base Solution (in 300 mL, pH 6.0).

| | Concentration |
|---|---|
| Component | (mg/L) |
| L-Alanine | 45 |
| B-Alanine | 45 |
| L-Arginine | 177.3 |
| L-Asparagine•H2O | 171.1 |
| L-Aspartic Acid | 133 |
| L-Cysteine•HCl•H2O | 351 |
| L-Glutamic Acid | 406.5 |
| L-Glutamine | 680 |
| Glycine | 750 |
| L-Histidine | 155 |
| L-Hydroxyproline | 65 |
| L-Isoleucine | 131 |
| L-Leucine | 131 |
| L-Lysine•HCl | 182 |
| L-Methionine | 149 |
| L-Ornithine-HCL | 84 |
| L-Phenylalanine | 165 |
| L-Proline | 115 |
| L-Serine | 525 |
| L-Threonine | 119 |
| L-Tryptophan | 102 |
| L-Tyrosine•2Na•2H2O | 112.6 |
| L-Valine | 117 |
| D-Glucose | 2000 |
| Inosine | 2.7 |
| Thymidine | 2.4 |
| Uridine | 2.4 |
| 1-Thioglycerol | 5.4 |
| DL-Carnitine-HCL | 1000 |
| Malic Acid | 670 |
| Oxaloacetic acid | 250 |
| Sodium Acetate•3H₂O | 25 |
| Sodium Pyruvate | 110 |
| Succinic acid | 60 |
| Potassium Chloride | 2000 |
| Magnesium sulfate Anhydrous | 600.6 |
| Sodium Chloride | 3200 |
| Sodium Phosphate Monobasic•H2O | 420 |

To this composition is added 111 mg/L of Calcium Chloride Anhydrous. Complete the preparation by following the Overall Instructions above.

“ZO Fortified media” refers to ZO media supplemented with insulin (5 μg/mL), a disaccharide such as trehalose (26.4 mM), L-alanyl-L-glutamine (ala-gln, 12 M), and L-ascorbic acid 2-phosphate sesquimagnesium salt hydrate (A2P, 0.08 M), with pH adjusted to 6.75, where the parenthetical values can be varied by about +/−50% in some embodiments, and the pH can be from about 6 to about 8. ZO Fortified media is prepared as follows, continuing from the Overall Instructions for preparing ZO media.

E. Prepare ZO Fortified from ZO Media (500 mL)

1) Take 248.75 mL ZO Media (2×) and add 248.75 mL MilliQ water

2) Add Aln-Gln, A2P Mag, and trehalose. Stir to dissolve

3) Add 2.5 mL human insulin (1 mg/mL stock prepared in PBS)

4) Adjust pH to 6.75

5) Filter sterilize with 0.2 um and store for use

### ZO Fortified (500 mL) Additives:

| | Concentration | |
|---|---|---|
| Component | (mg/L) | mg |
| L-Ascorbic acid 2-phosphate | 23.13 | 11.565 |
| sesquimagnesium salt hydrate | | |
| Trehalose | 10000 | 5000 |
| Alanyl-glutamine | 2600 | 1300 |
| Human Insulin | 5 μg/mL | 2.5 mL |

“ZB media” refers to ZO Fortified media supplemented with spermidine at a concentration of about 0.05 μM to about 10 μM, typically about 1 μM. ZB media is prepared as follows, continuing from the Overall Instructions for preparing ZO media and ZO Fortified media.

F. Prepare and Use ZB Media
Spermidine can be unstable; prepare small batches. Generally use ZO Fortified and supplement with 1 μM spermidine trihydrochloride at time of use.
1) Prepare a 1 mM stock of spermidine trihydrochloride, aliquot and freeze. Supplement ZO Fortified with 1 μM (1:1000 dilution of stock) at time of use.

“ZW media” refers to a chemically defined minimal media developed by Wyss (Wyss, Exp. Cell Res., 1982, 139, 297-307). In various embodiments, the additives of ZO Fortified that are added to ZO media can be added to ZW media to provide an additional media (ZW Fortified). Furthermore, a polyamine, as described above for ZB media, can be added to ZW Fortified media to provide an additional media for use in the methods described herein. ZW media includes the following components.

### Composition of Medium ZW (in Mg/L):

| | |
|---|---|
| L-Alanine | 45 |
| ,B-Alanine | 45 |
| L-Arginine | 174 |
| L-Asparagine | 150 |
| L-Aspartic acid | 133 |
| L-Cysteine-HCl | 315 |
| L-Glutamine | 680 |
| L-Glutamic acid | 294 |
| Glycine | 750 |
| L-Histidine | 620 |
| L-Hydroxyproline | 65 |
| L-Isoleucine | 131 |
| L-Leucine | 131 |
| L-Lysine-HCl | 182 |
| L-Methionine | 149 |
| L-Ornithine-HCl | 84 |
| L-Phenylalanine | 165 |
| L-Proline | 115 |
| L-Serine | 525 |
| L-Threonine | 119 |
| L-Tryptophane | 102 |
| L-Tyrosine | 90 |
| L-Valine | 117 |
| Glucose | 2,000 |
| Inosine | 2.7 |
| Thymidine | 2.4 |
| Uridine | 2.4 |
| 1-Thioglycerol | 5.4 |
| DL-Carnitine-HCl | 1,000 |
| Glutathione, reduced | 50 |
| Malic acid | 670 |
| Oxaloacetic acid | 250 |
| Sodium acetate × 3 H₂O | 25 |
| Sodium pyruvate | 110 |
| Succinic acid | 60 |
| CaCl₂ × 2 H2O | 147 |
| KCl | 2,000 |
| MgS0₄ × 7 H2O | 1,230 |
| NaCl | 3,200 |
| NaH₂PO₄ × H2O | 420 |
| Minor salts | |
| CuS0₄ | 0.001 |
| FeSO₄ × 7 H₂O | 1 |
| MnSO₄ × 7 H₂O | 1 |
| ZnSO₄ × 7 H₂O | 1 |
| Vitamins | |
| Biotin | 0.05 |
| Calcium pantothenate | 5 |
| Choline chloride | 50 |
| Folic acid | 0.05 |
| m-Inositol | 50 |
| Niacinamide | 0.5 |
| Pyridoxal-HCl | 5 |
| Riboflavin | 0.01 |
| Thiamine-HCl | 5 |
| Vitamin B 12 | 0.05 |
| Lipids | |
| Cholesterol | 0.01 |
| Linoleic acid | 0.01 |

pH adjusted to 6.6 with 1N NaOH.
Final osmolarity, 250 mOsm.

As would be readily recognized by one of skill in the art, one or more components of ZO media or ZW media can be omitted, while still maintaining an effective growth media, depending on the species to be grown and the objective of the culture. Thus, various media lacking a component above are included within the scope of the invention, where the resulting media can be described as ZO media or ZW media lacking X or component X, where X is the omitted ingredient of ZO media or ZW media. In other embodiments, the formulation of ZO media or ZW media will be substantially (e.g., within a reasonable experimental error, such as +/−2-5 wt. %) the ZO media described above.

As used herein, the term “polyamine” refers to an organic compound that includes at least two nitrogen atoms in the molecule. The polyamine can be a diamine such as ethane-1,2-diamine, propane-1,3-diamine, putrescine (butane-1,4-diamine), pentane-1,5-diamine, hexane-1,6-diamine, and the like, including compounds with branched alkyl chains and positional isomers with respect to the amine groups, optionally including substituent groups, such as for ornithine. In some embodiments, the diamine is a compound of the formula NH₂—R—NH₂ where R is alkyl, such as a (C₁-C₁₂)alkyl, straight chain or branched. The polyamine can also be a triamine such as spermidine and other polyamines of the formula NH₂—R—NH—R—NH₂ where each R is independently alkyl, such as (C₁-C₁₂)alkyl, straight chain or branched. The polyamine can also be a tetraamine such as spermine and other polyamines of the formula NH₂—R—NH—R—NH—R—NH₂ where each R is independently alkyl, such as (C₁-C₁₂)alkyl, straight chain or branched. The polyamine can also be a polymer such as polyethylenimine (PEI), which polymers can have a molecular weight of about 0.6 kDa to about 250 kDa. A variety of polyamines are available from commercial suppliers such as Sigma-Aldrich, Acros Organics, and Polysciences, Inc., or they can be prepared using techniques well known to those of skill in the art.

The phrase “long-term growth” of a cell line, such as a Drosophila cell line, refers to the growth and proliferation of a cell line through at least three culture passages. Cell lines can be cultured, for example, according to instructions from the Drosophila Genomics Resource Center (DGRC).

As used herein, an “agent”, “candidate compound”, or “test compound” is an organic compound or inorganic salt that is of interest for being tested for relevance in a biological system, for example, as a growth factor or as a growth inhibitor. The test compound can be, for example, a small molecule, nucleic acid (e.g., DNA and RNA), carbohydrate, lipid, protein, peptide, peptidomimetic, or a drug. The test compounds are typically small molecules, such as organic compounds having a molecular weight of less than about 800 Da, less than about 750 Da, or less than about 600 Da.

### Inverse Screen to Design a Chemically Defined Medium Supporting Long-Term Growth of Insect Cell Lines

The invention provides a method for translating compound phenotypic screen scores to biological target scores and enriched ontology terms important for the process of interest. This method relies on the use of a compound-protein interaction database, the Search Tool for Interactions of Chemicals (STITCH). Our target analysis approach can be extended to develop rationally designed compound libraries targeting a large or the maximum possible genome coverage (or target-of-interest coverage) in a small or the minimum library size based on interactions cataloged in STITCH.

Library diversity and coverage is a major concern when compiling compound libraries for high-throughput screening by pharmaceutical companies and academic labs alike. Most frequently, rational methods for diversifying chemical libraries by companies rely on diversifying compound properties, especially physiochemical and structural properties like appendages, skeletal, stereochemical, and functional groups. These methods are especially appropriate for pharmaceutical companies interested in new drug discovery. However, the use of compound libraries for inverse screening—that is, screening compounds with known targets for phenotypic effects to elucidate targets important for a given biological process—is becoming more widespread.

One of the benefits to using such an inverse drug screen as opposed to a genetic loss-of-function screen is efficiency: one molecule can simultaneously probe multiple putative targets, saving time and money. Design of small molecule libraries for the inverse screen described herein should rely on different criteria than those used to create libraries for drug discovery; in particular, inverse drug-screening libraries should be designed to maximize target coverage while minimizing compound number. Further, redundancy should be considered and tackled systematically. Libraries can also be designed to specifically cover targets of interest only in order to maximize efficiency directly.

Described herein is a method for rationally designing compound libraries based on cataloged drug-protein interactions in STITCH and other databases. Briefly, a list of proteins of interest (or protein products of the entire proteome) would be generated and the STITCH database would be queried to identify compounds targeting those proteins of interest and the associated interaction score. Optimization strategies would then be applied to generate the smallest library list that probes all proteins of interest with a minimum interaction score. “Smart pooling” strategies developed for multi-compound high throughput screening could be adapted for this purpose (e.g. shifted transversal design), as the basis is similar except targets would be pooled as opposed to compounds.

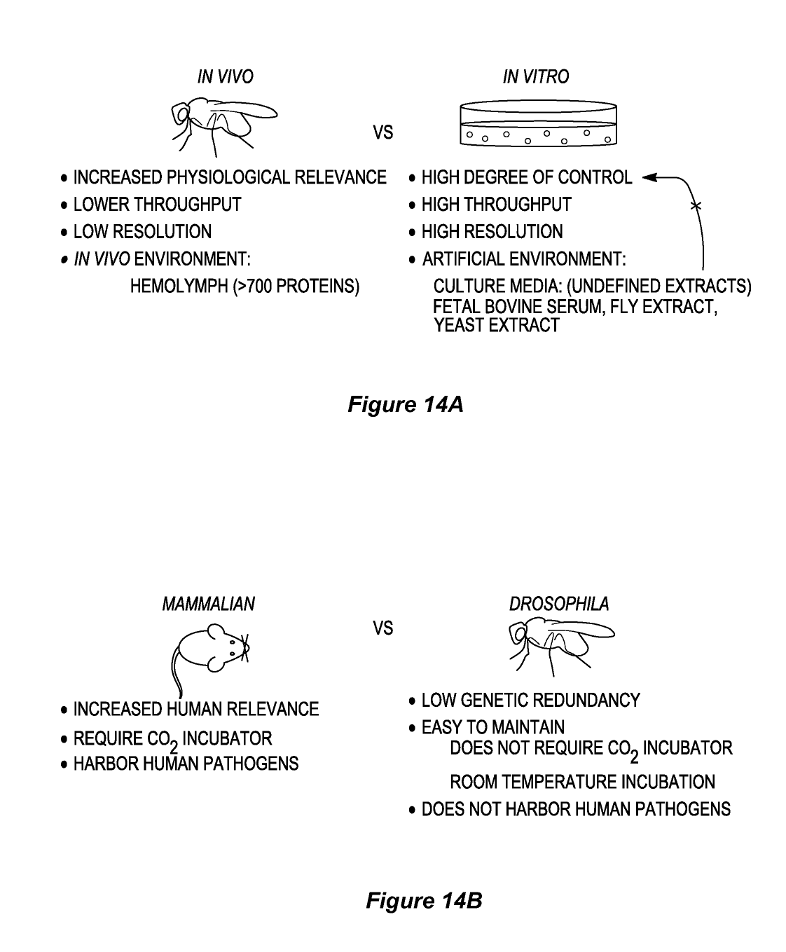

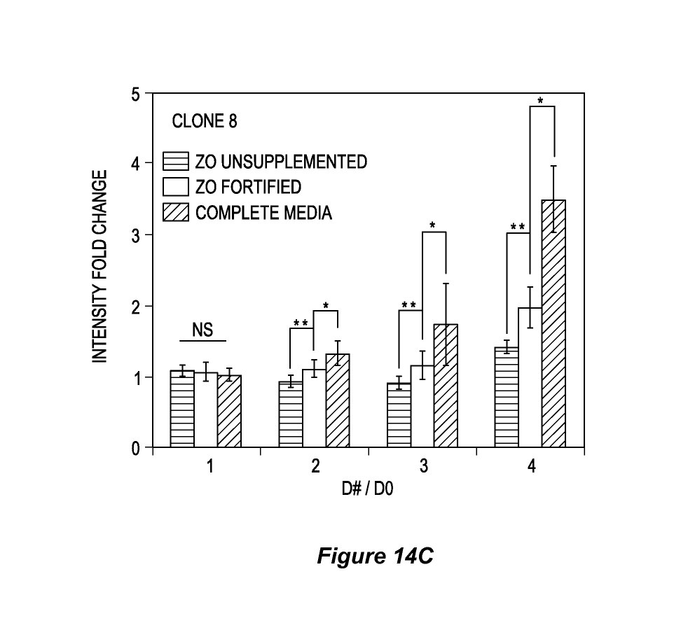

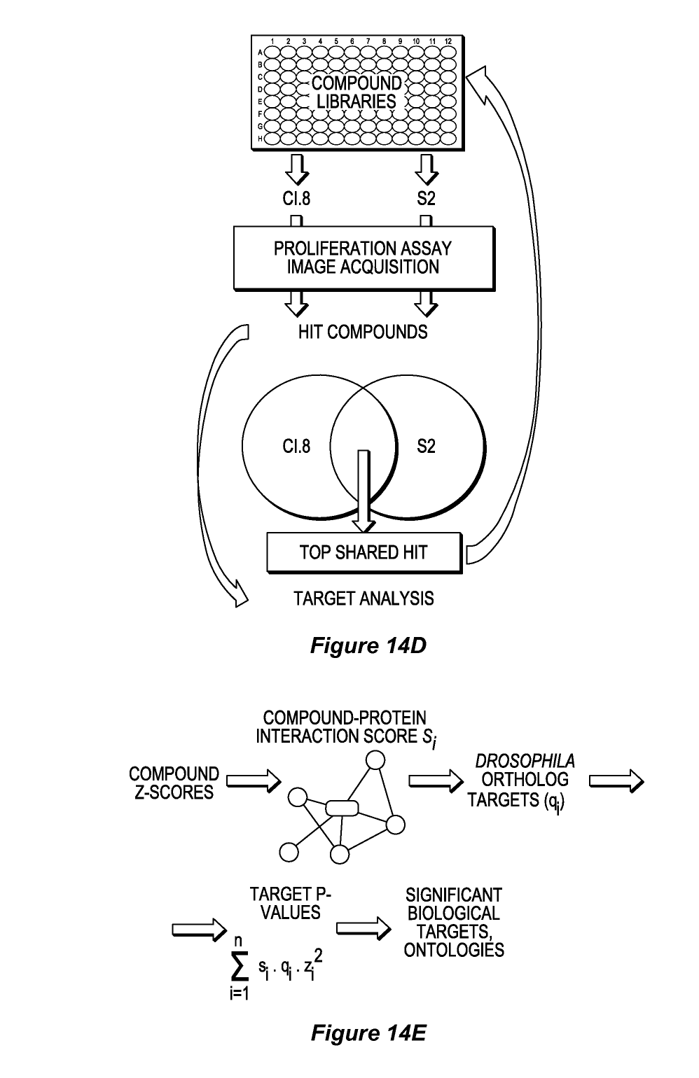

Provided herein is the demonstration of a high-throughput inverse drug-screening platform to identify novel compounds and genetic targets important for proliferation of Drosophila cells. By systematically identifying and scoring protein targets of the screened compounds, we can identify genes and pathways in addition to compounds important for growth. The approach harnesses a chemical-protein interaction database to “translate” cumulative small molecule scores to gene target scores to elucidate targets with small effects (e.g. small effects from multiple compounds with same target) (FIG. 14E). This approach is an improvement over traditional methods where only “hit” compounds' targets are investigated, and can be applied to screens in other model organisms for which databases are available. The pipeline can also be expanded to identify compound synergies that can be exploited to design CDM capable of supporting long-term growth of multiple insect cell lines, such as Drosophila cell lines (FIG. 14D). The protocol can be used as a template for the rational design of media, to identify growth-promoting factors, and implicate signaling pathways important for growth.

In this proof-of-principle screen, we focus on two standard Drosophila cell lines, the adherent Clone 8 (C1.8), which has previously been used to identify novel insect-specific growth factors, and S2-DRSC (S2), which is frequently used for recombinant protein production and grows in suspension (Schneider, J. Embryol. Exp. Morphol., 1972, 27, 353-365). The screen led to the identification of multiple candidate molecules relevant for stimulating growth and viability of both cell lines. In particular, the pilot screen revealed polyamines as a key component of a CDM for Drosophila cells, and sufficient for enabling long-term growth of C1.8 and Kc167 cells without requiring any weaning of the cells from sera. To our knowledge this is the first successful attempt to harness a small molecule screen to systematically define the minimal requirements for long-term Drosophila cell growth in a chemically defined environment. The media and experiments are further described below in Examples 1-3.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Polyamines as Key Molecules for Long-Term Maintenance of Drosophila Cells in a Chemically-Defined Medium

Drosophila has lent itself to a wide range of applications as a versatile genetic model system to understand the development and physiology of multiple tissue types, including epithelial tissues. The relative lack of in vitro methods for Drosophila cell and tissue culture compared to mammalian cell culture technology has begun to limit the potential of Drosophila research as a biochemical model system for studying human diseases. Accordingly, new methods are needed to enhance in vitro Drosophila cell research. Examples of areas that require work include the establishment of more efficient cloning techniques and the development of completely chemically defined media.

Described herein is an adaptive screening strategy carried out on two cell lines to identify compounds and compound synergies important for stimulating proliferation of Drosophila cells in a chemically defined medium. A high throughput screening pipeline enabled us to identify not only novel compounds important for stimulating proliferation of Drosophila cells, but also potential compound synergies that can be exploited to design a chemically defined media capable of supporting long-term growth in multiple Drosophila cell lines. In this example we have optimized the statistical design and analysis of the experiments. The screening protocol can be used as a template for the rational design of minimal media or to identify growth-promoting factors.

In this proof-of-principle screen, we focused two cell lines: C1.8, which is well characterized and has been previously used to identify novel insect-specific growth factors and as a proxy for optimizing short-term culture of primary wing disc explants; and an embryonic cell line with macrophage-like lineage, S2, which is well characterized and frequently used for recombinant protein production. We have identified test molecules relevant for stimulating growth/longevity of both cell lines.

In our pilot screen, we identified polyamines as an important component of chemically defined media for Drosophila cells. We also identified Bruton's tyrosine kinase (BTK) inhibition as another potential avenue to stimulate proliferation via inhibition of Wnt antagonism by BTK. Our cell-based method is extensible to the screening of recombinant growth factors and can be extended to organ culture studies. Expansion of our initial screen enables the identification of a combination of small molecules that can support the maintenance and generation of Drosophila cell lines in a chemically defined medium. Such a medium would facilitate many biochemical studies such as stable isotope labeling by amino acids in cell culture (SILAC) that are currently hindered by the complexity and undefined nature of serum-containing medium. Additional information, techniques, and methods are described by Burnette and coworkers in Mol. BioSyst., 2014, 10, 2713-2723 and its electronic supplementary information, which are incorporated herein by reference.

Materials and Methods.

Cell Culture.

C1.8 and S2 cells were expanded in optimized serum-containing media, C1.8 media and S2 media respectively. C1.8 media is based on M3 media (Sigma) supplemented with 2% FCS, 5 μg/mL insulin, and 2.5% volume fly extract. S2 media is based on Schneider's media (Gibco) supplemented with 10% FCS. Cells were cultured according to instructions from the Drosophila Genomics Resource Center. All cells were maintained at 25° C. in a humidified incubator.

Basal Media and “ZO Fortified”.

We selected Wyss' ZO medium for Drosophila cells as the base of our media as it represents the previous effort to create a chemically defined medium for Drosophila. ZO media was initially acquired commercially (Sweden National Veterinary Institute) but is currently prepared in our lab in small batches as described by Wyss with similar results (Wyss, Exp. Cell Res., 1982, 139, 297-307; Wyss, Exp. Cell Res., 1982, 139, 309-319).

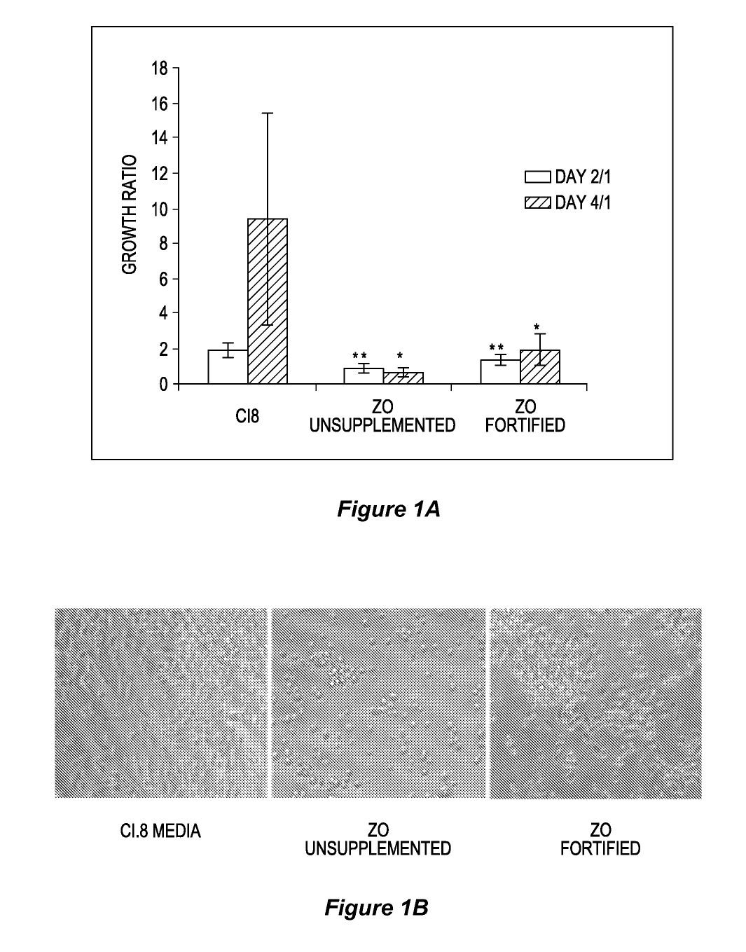

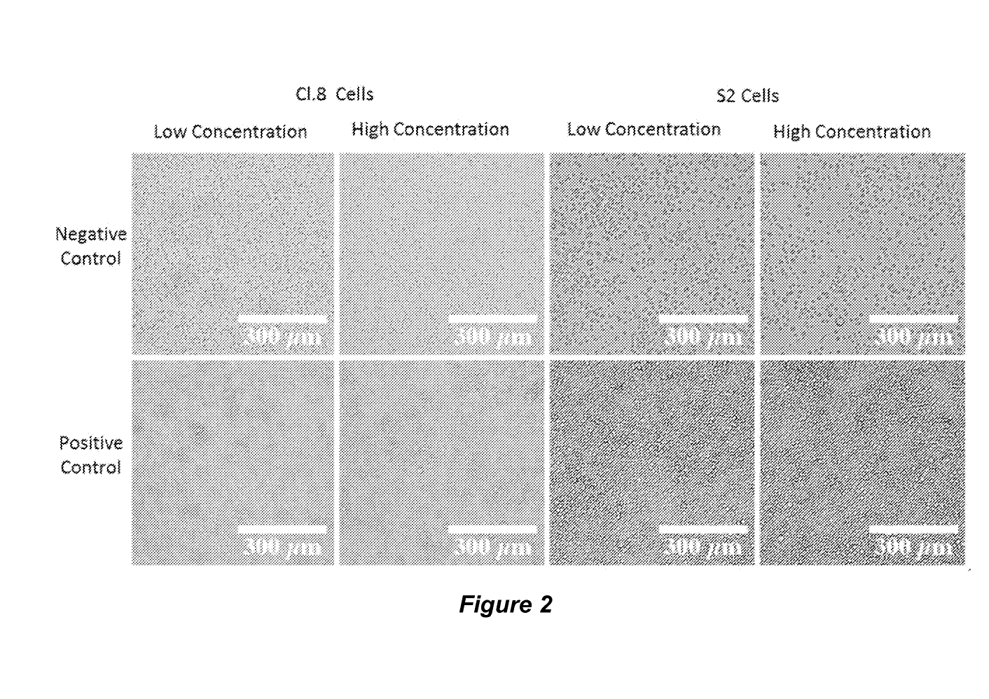

During the first phases of testing, we tested compound candidates from the literature for their effect on proliferation. The CyQUANT® Direct Proliferation Assay was used to determine cell number based on fluorescent intensity from DNA content (FIG. 1). Using this approach we developed an intermediate “ZO Fortified” medium, made up of ZO media supplemented with insulin (5 μg/mL), trehalose (26.4 μM), alanyl-glutamine (12 μM), and L-ascorbic acid (0.08 μM). Whereas ZO unsupplemented does not support proliferation of C1.8 cells, ZO Fortified supports C1.8 short-term cell growth and initial attachment, as can be seen in FIG. 2A. ZO Fortified is capable of supporting initial attachment of C1.8 cells (FIG. 2B) and growth through at least 3 passages.

Medium-Throughput Drug Screen.

To more efficiently identify compounds important for cell growth in serum-free media, we selected five small molecule libraries for high-throughput assay development and pilot screening. These libraries come in a convenient 96-well format in DMSO at 10 μM. Because one of the primary applications of libraries such as these is cancer-targeting therapy, the majority of these compounds are likely growth inhibitors rather than stimulators. However, network analysis of growth inhibitors' targets may provide important insights into which pathways are important to modulate in this CDM. To increase our potential of finding positive hits, we created a custom compound library consisting of compounds that can be important for cell growth in CDM or which may be deficient in ZO medium (Table A).

TABLE A
Custom library compounds.

| Compound | Concentration | Solvent |
|---|---|---|
| 1-Thioglycerol | 50 mM | EtOH |
| 20-Hydroxyecdysone | 1 pg/mL | DMSO |
| 4-Aminobenzoic Acid | 10 mM | DMSO |
| 8-Br-cAMP | 10 mM | DMSO |
| 8-pCPT-CGMP | 10 mM | DMSO |
| Aminoguanidine hemisulfate salt | 40 mM | DMSO |
| Aminophylline | 10 mM | DMSO |
| Sigma A1345 Antioxidant Supplement | 1000x | — |
| bpV(phen) | 10 nM | DMSO |
| D-NAME | 1M | DMSO |
| DIDS | 10 mM | DMSO |
| Dipyridamole | 10 mM | DMSO |
| Sigma F7050 Fatty Acid Supplement | 100x | — |
| Ferritin | 10 mM | DMSO |
| Folic Acid | 10 mM | DMSO |
| Glybenclamide | 10 mM | DMSO |
| Guanosine | 10 mM | DMSO |
| Inosine | 10 mM | DMSO |
| L-Arginine | 10 mM | DMSO |
| L-Ascorbic acid 2-phosphate | | |
| sesquimagnesium salt hydrate | 10 mM | DMSO |
| L-Cysteine hydrochloride | | |
| aminophosphate | 10 mM | DMSO |
| L-Glutathione Reduced | 10 mM | DMSO |
| L-Lysine Monohydrochloride | 10 mM | Water |
| L-Methionine | 10 mM | Water |
| L-Proline | 10 mM | DMSO |
| L-Tyrosine Disodium Salt Hydrate | 10 mM | Water |
| Sigma L0288 Lipid Mix 1 | 1000x | Water |
| myo-Inositol | 10 mM | DMSO |
| N-Acetyl Glucosamine | 10 mM | DMSO |
| Nicotinamide | 10 mM | DMSO |
| Nicotinic Acid | 10 mM | DMSO |
| Oxaloacetic Acid | 1.9M | DMSO |
| Phorbol 12-mirisitate 13-acetate | 3 mM | DMSO |
| Phorbol-12,13-diacetate | 2 mM | Water |
| Sigma P8483 Polyamine supplement | 500x | H₂0 |
| Potassium chloride | 10 mM | DMSO |
| Putrescine dihydrochloride | 10 mM | DMSO |
| Putrescine dihydrochloride | 100 mM | DMSO |
| Putrescine dihydrochloride | 10 mM | DMSO |
| SITS | 10 mM | DMSO |
| Sodium Chloride | 10 mM | DMSO |
| Spermidine | 10 mM | DMSO |
| Spermine | 10 mM | DMSO |
| Tamoxifen | 10 mM | DMSO |
| Tocopherol | 10 mM | EtOH |
| Mediatech 99182-Cl Trace A | 1000x | — |
| Mediatech 99175-Cl Trace B | 1000x | — |
| Mediatech 99176-Cl Trace C | 1000x | — |
| Sigma 903244 Vanderzant Vitamin | | |
| Mixture | 10 mg/mL | DMSO |
| Vitamin D2 | 0.13 mM | EtOH |
| Vitamin K1 | 2.2 mM | EtOH |
| Vitamin K1 | 0.11 mM | EtOH |
| Selleck S1049 Y27632 ROCK inhibitor | 200 uM | PBS |

During previous investigations we found working concentrations for some of these compounds. The custom library stocks were therefore prepared either at 10 μM (for un-tested compounds) or 10,000× their working concentration (based on preliminary testing). The vast majority of these compounds stocks were prepared in DSMO, but some were prepared in water or ethanol based on solubility (see Table A for details). These 6 libraries served as the basis for our screening efforts.

Five stock plates of randomized compounds (410 total) were prepared at 75 μM in water (1:133 dilution) using an automated liquid handler (Eppendorf EPMotion 5075). Strategically located negative control wells were incorporated into the stock plates by dosing wells with DMSO (1:133, carrier only control). Randomly placed positive control wells were also included by dosing wells with DMSO (1:133); for flexibility, positive controls were prepared in the working stock. Working stocks plates at 2 and 20 μM (2×) were then prepared in ZO Fortified by diluting the 5 stock plates 1:3.75 (high concentration working stock) and then serially diluting 1:10 (low concentration working stock). Positive control wells were prepared by replacing 10% of the media with 100% fly extract (FEX).

Cells were rinsed three times in PBS and seeded at a concentration of 50,000 cells/well (50 μL) in ZO Fortified (primary screen) or ZO Fortified supplemented with 2 μM spermidine (secondary screen) in 10 plates. Working stocks, 50 μL, were then immediately added on top of the cells (day 0). Plates were incubated at 25° C. for four days. On the fourth day, plates were imaged at 10× magnification on a confocal microscope using automated screen acquisition (Andor Spinning Disc Confocal Microscope, Metamorph Software). CyQUANT® Direct Proliferation Assay (Invitrogen) was then added (0.25× final conc.) and fluorescent intensity was measured with a plate reader (Biotek Synergy H2).

Cell Culture in Chemically Defined Medium.

Cells were rinsed 3× in PBS and then seeded at high confluency in ZO Fortified and ZB Media (ZO Fortified supplemented with 1 μM spermidine). Upon reaching confluency, cells were passaged 1:2 retaining half of the spent media.

Results.

Primary Screen in ZO Fortified.

Strictly standardized mean difference (SSMD) scores for classifying assay quality were calculated with respect to negative controls on a plate-wise basis, with the average assay quality being excellent for all both screened concentrations (Table 1).

TABLE 1
SSMDs and classifications for Cl.8 and
S2 primary screens (ZO Fortified).

| | |
|---|---|
| Low Concentration | High Concentration |

| | SSMD | Classification | SSMD | Classification |
|---|---|---|---|---|
| Cl8 | 24.8 | Excellent | 17.6 | Excellent |
| S2 | 12.7 | Excellent | 10.8 | Excellent |

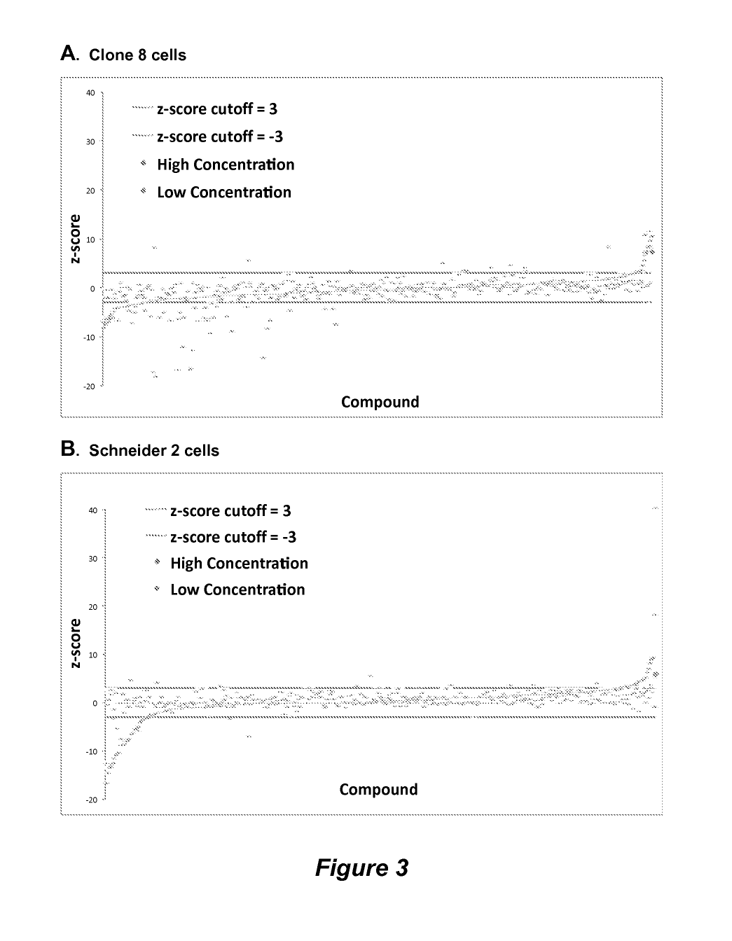

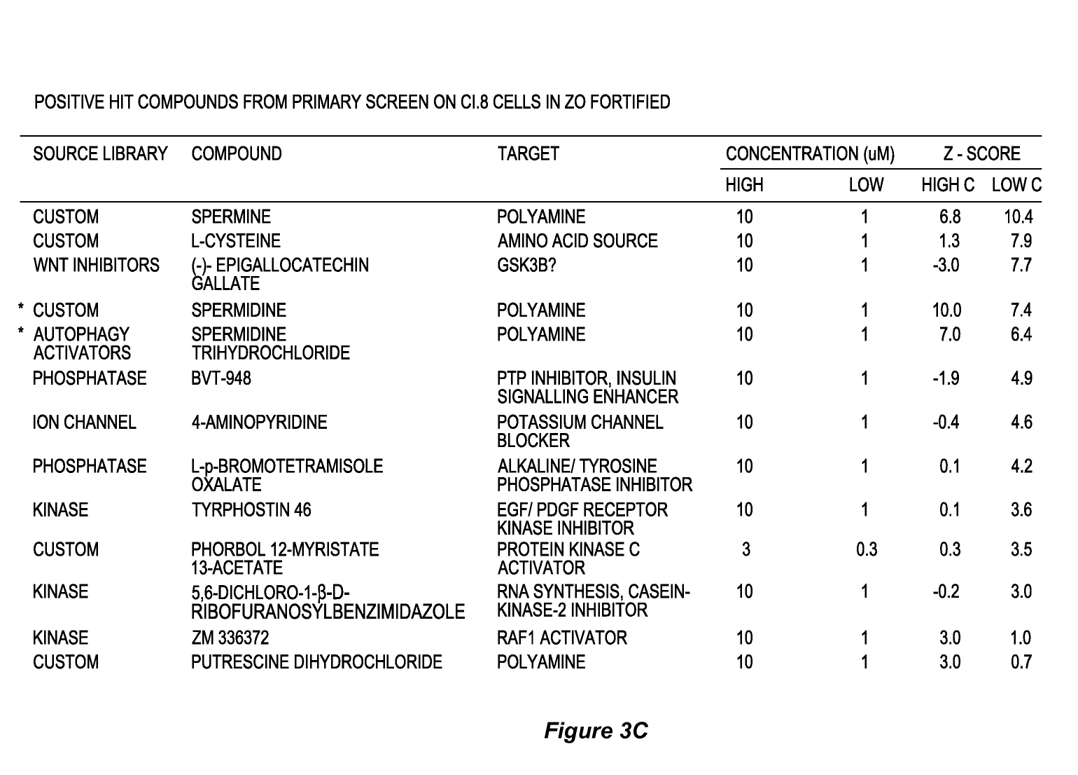

Representative positive and negative control wells can be found in FIG. 2. Compound z-scores were calculated similarly, hit cutoffs of ±3 were established visual inspection of confocal images was used to remove false-positive hits. This analysis yielded 13 C1.8 and 23 S2 positive hit compounds and 80 C1.8 and 36 S2 negative hit compounds (see FIG. 3 and Table A (above)).

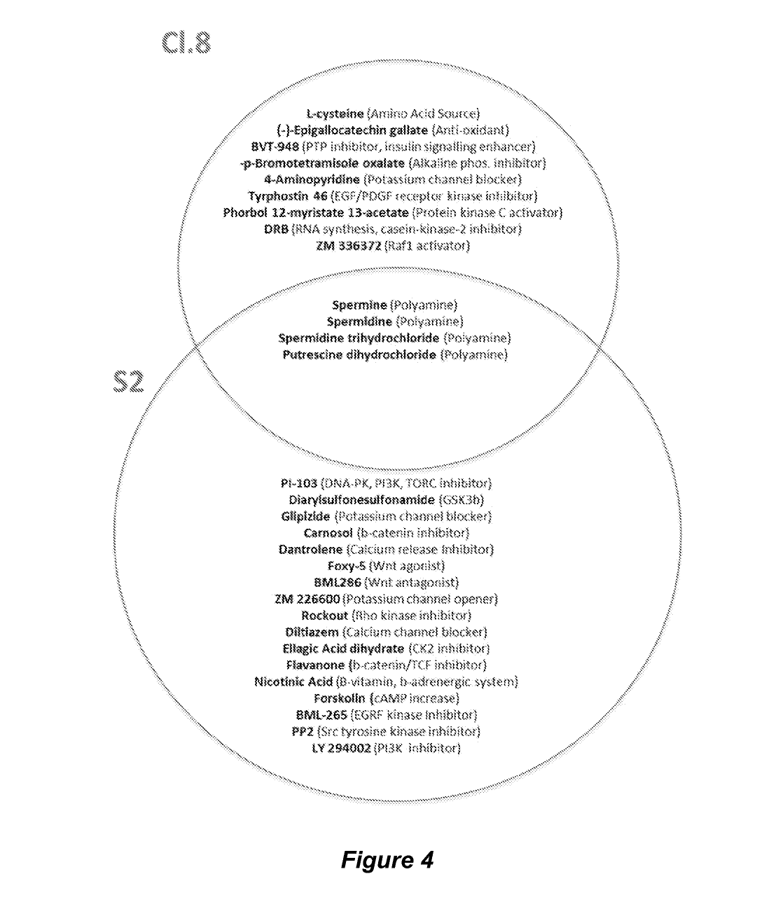

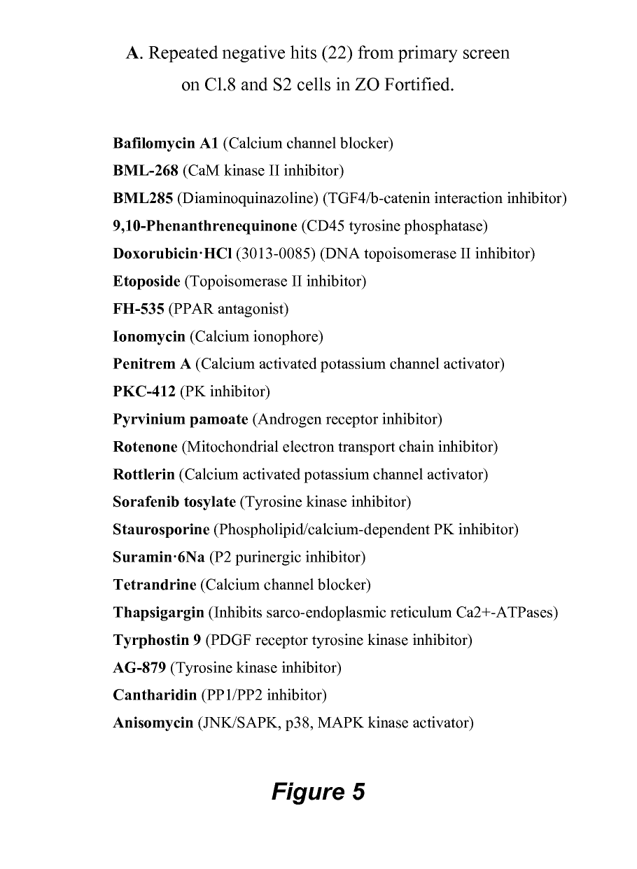

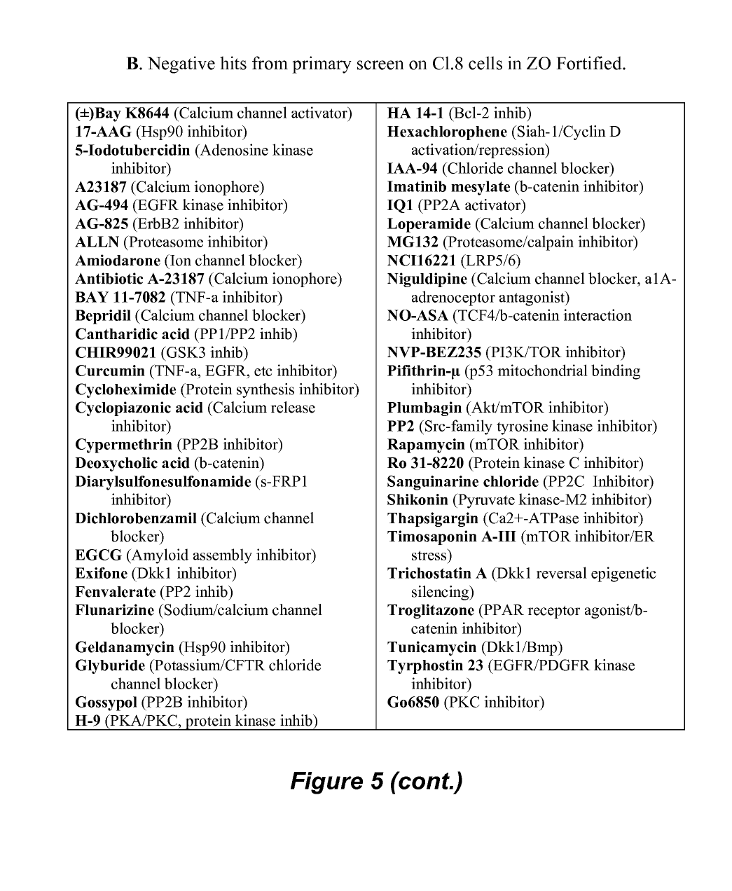

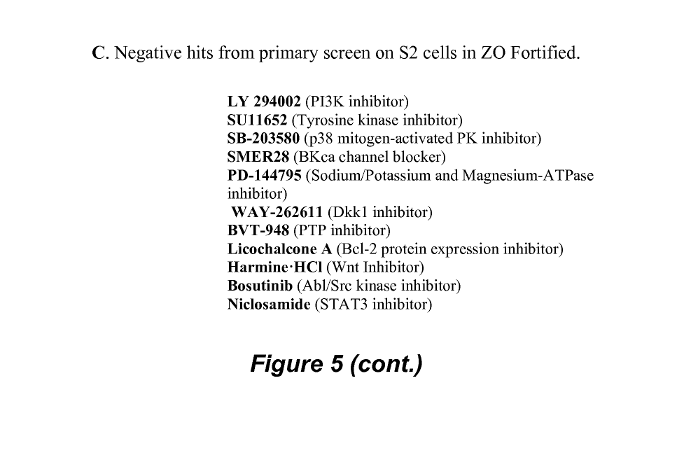

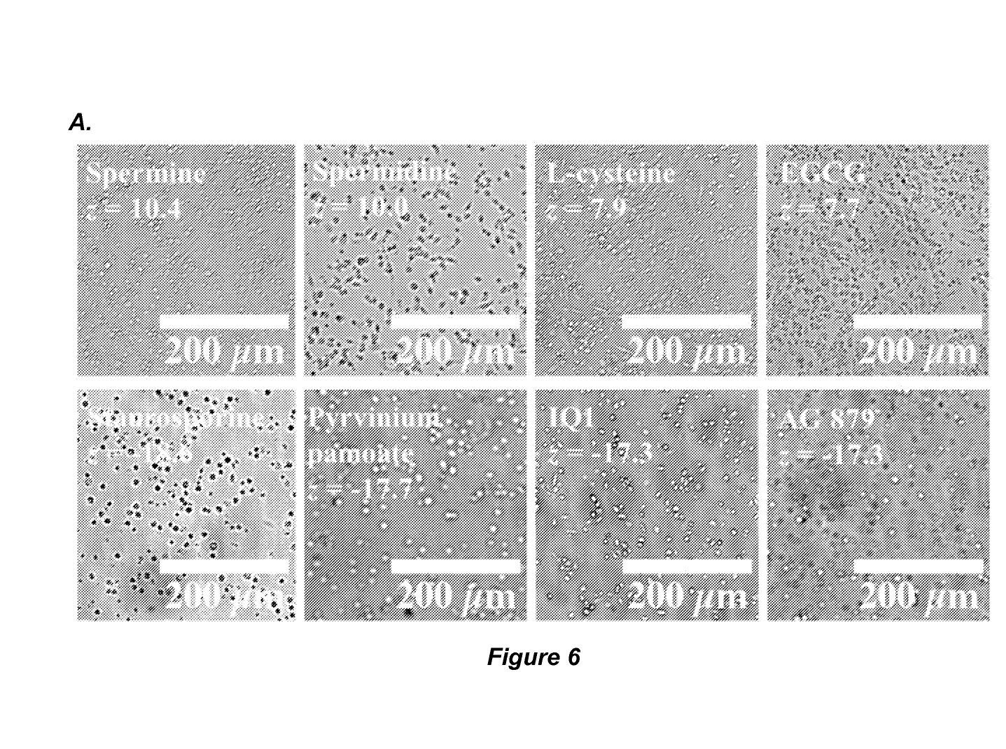

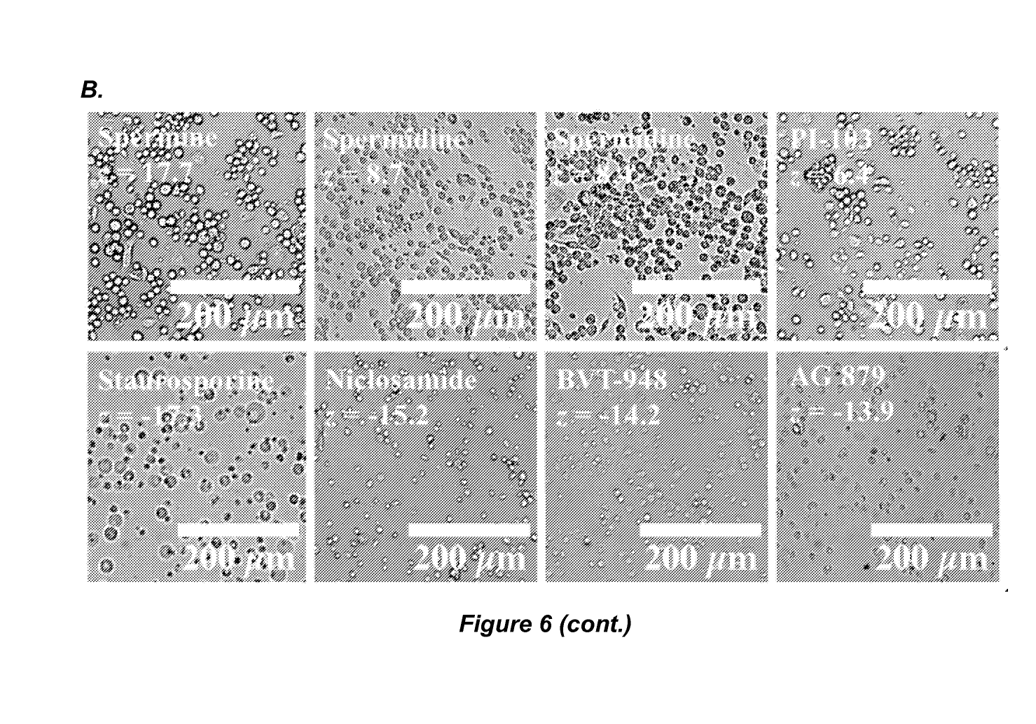

Of the positive hit compounds, four were repeated between the two cell lines (FIG. 4). All of the repeated hits were polyamines: spermine, spermidine, and putrescine, with spermine and spermine representing the two strongest positive hits in both cell lines. Due to the lower cost of spermidine and our desire for an economical CDM, we incorporated 1 μM spermidine into ZO Fortified, newly termed “ZB Media,” and began to passage C1.8 and S2 cells in this media to determine its ability to support long-term growth. Of the negative hits, 22 were repeated between the two cell lines (FIG. 5). Images of the four strongest positive and negative hits from each cell line can be found in FIG. 6.

Cell Culture in Chemically Defined Medium.

After 3 passages, unsupplemented C1.8 cells' proliferation rates significantly decreased compared to cells supplemented with spermidine. Spermidine supplemented C1.8 cells have reached >15 passages without any signs of changed growth dynamics whereas the unsupplemented remain on passage 4. Upon reaching high density, cells were passaged 1:2 retaining half of the spent media. Spermidine supplemented S2 cells are currently on passage 4 whereas unsupplemented S2 cells have yet to make it to pass 2.

Secondary Screen in ZB Media.

SSMD scores for classifying assay quality were calculated with respect to negative controls on a plate-wise basis, with the average assay quality being excellent for both concentrations (Table 2).

TABLE 2
SSMDs and classifications³⁰ for Cl.8
and S2 secondary screens (ZB Media).

| | |
|---|---|
| Low Concentration | High Concentration |

| | SSMD | Classification | SSMD | Classification |
|---|---|---|---|---|
| Cl8 | 4.3 | Good | 14.7 | Excellent |
| S2 | 3.3 | Good | Good | Excellent |

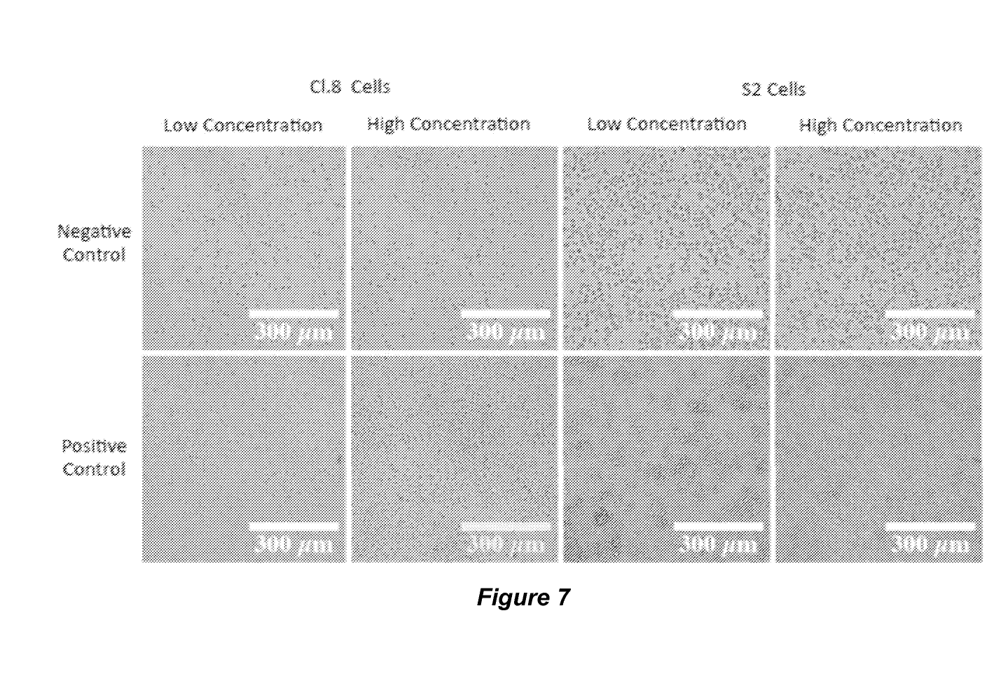

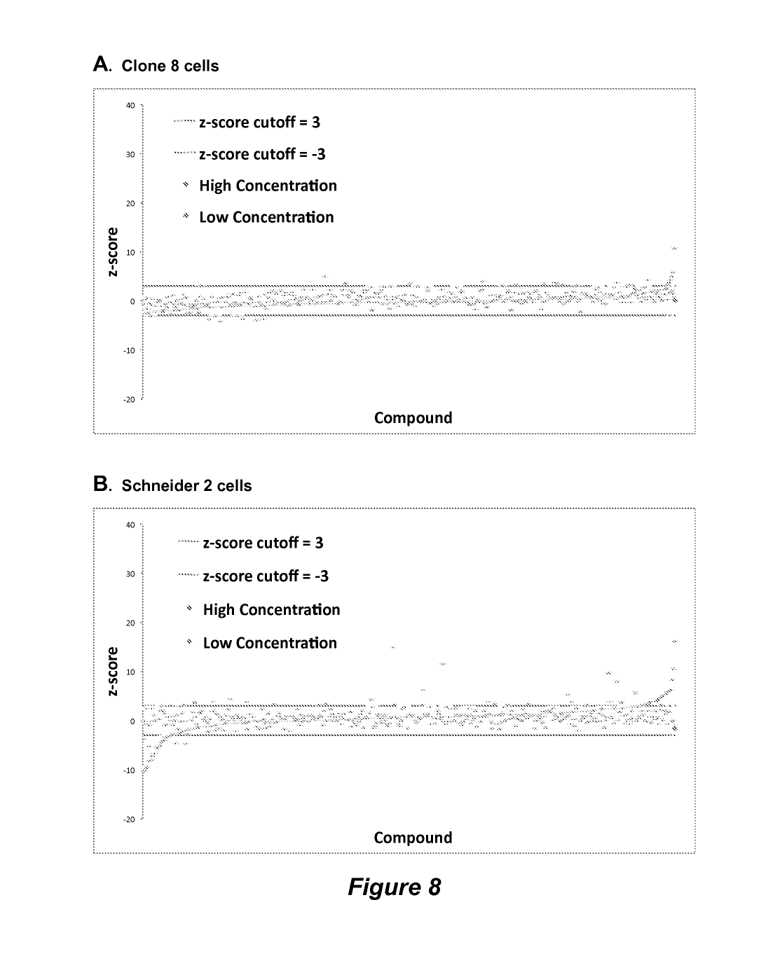

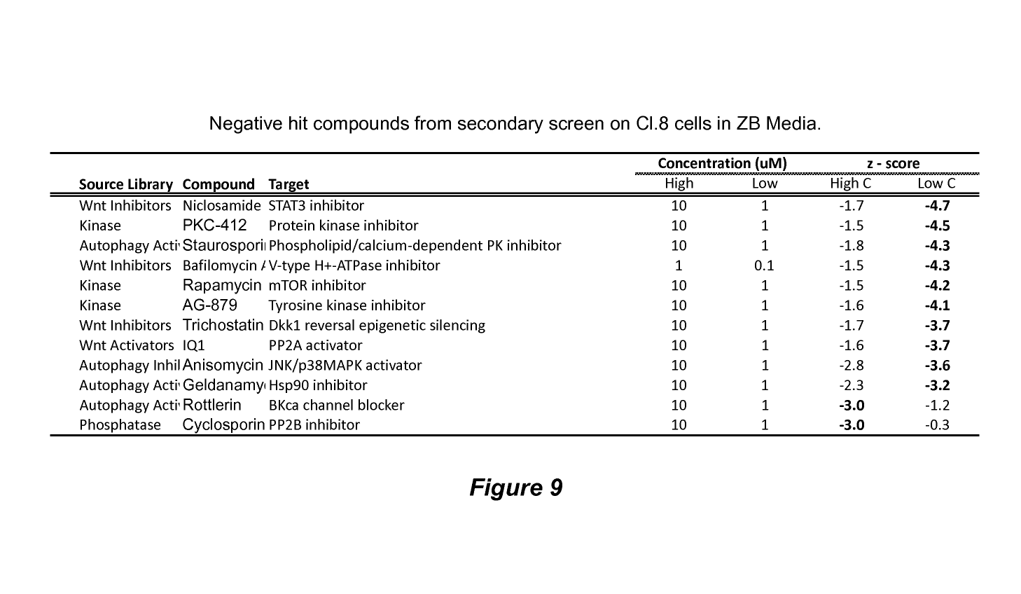

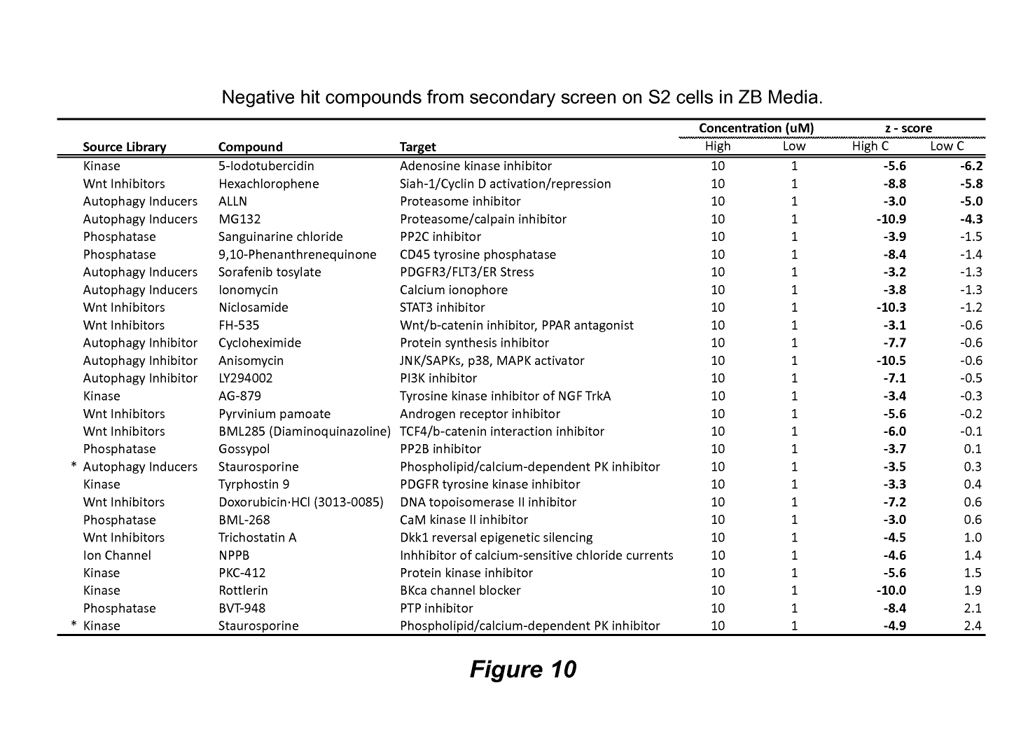

Representative positive and negative control wells can be found in FIG. 7. Compound z-scores were calculated similarly, hit cutoffs of ±3 were established visual inspection of confocal images was used to remove false-positive hits. This analysis yielded 8 C1.8 and 27 S2 positive hit compounds and 12 C1.8 and 27 S2 negative hit compounds (FIGS. 8-10).

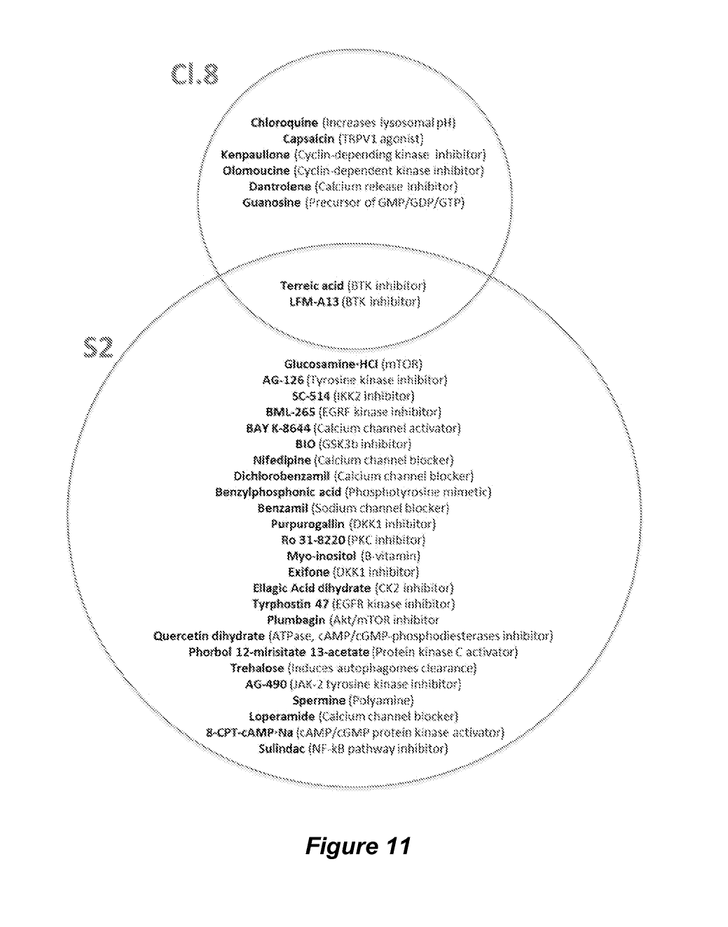

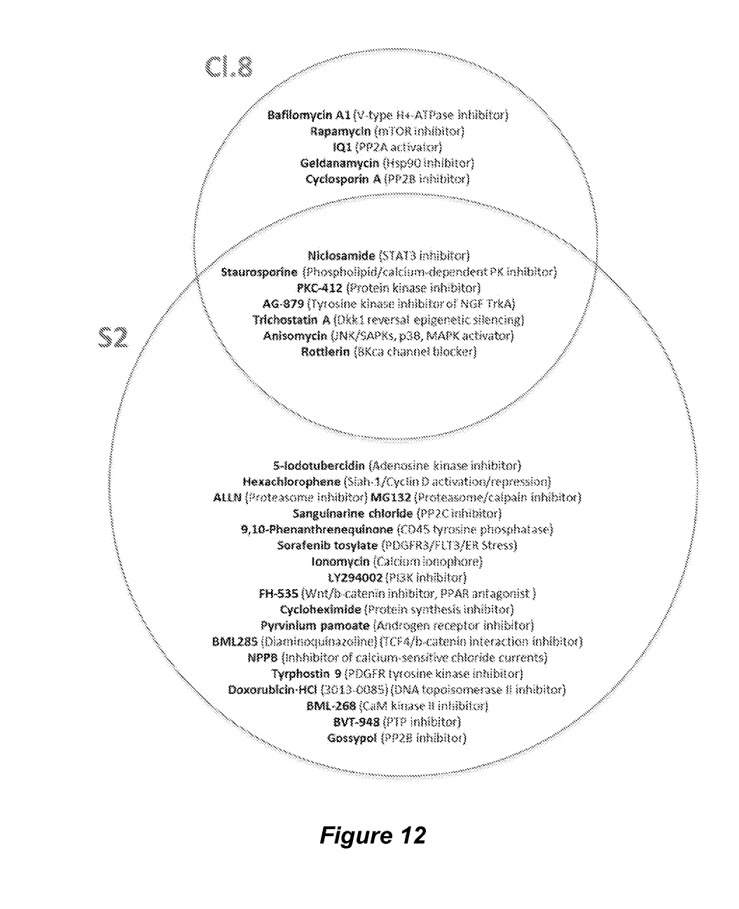

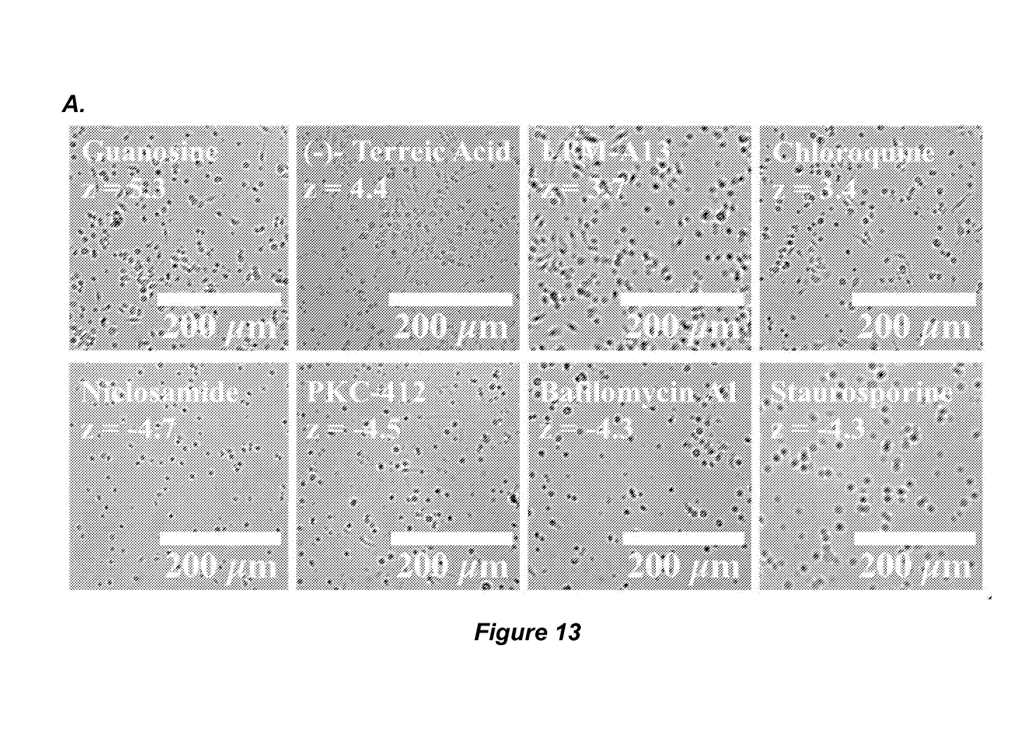

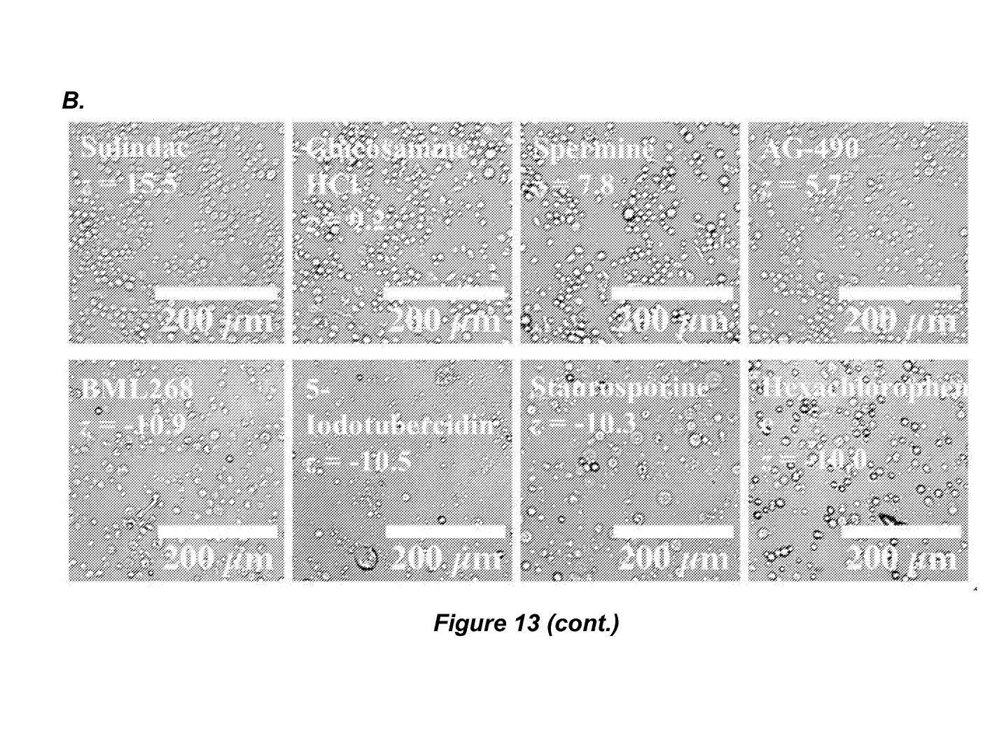

Of the positive hit compounds, 2 were repeated between the two cell lines (FIG. 11), LFM-A13 and terreic acid, both inhibitors of Bruton's tyrosine kinase, whose inhibition has been shown to increase Wnt signaling via inhibition of antagonism by BTK, and whose Drosophila homolog has been shown to be required for adult survival and male genital formation. Of the negative hit compounds, 7 were repeated between the two cell lines (FIG. 12). Images of the four strongest positive and negative hits from each cell line can be found in FIG. 13.

Spermidine has thus been shown to be beneficial for maintaining both C1.8 and S2 cells in our chemically defined media, and sufficient for C1.8 cells. Spermidine promotes stress resistance and longevity in the Drosophila. The inhibition of Bruton's tyrosine kinase can also serve as an avenue for increasing cell proliferation in this chemically defined medium via increased Wnt signaling.

Accordingly, this optimized, serum-free media for cell proliferation and primary cell line establishment is a valuable tool for insect cell line developmental research. The media can also be used for production of recombinant therapeutic proteins. The pipeline can be used to custom design culture systems for the rapid creation of new cell lines from primary tissues by stimulating the relevant pathways pharmacologically rather than by traditional genetic methods. The media produced by the pipeline can be of great use to further insect cell culture methods, improve conditions for industrial production of recombinant proteins from insect cells, increase success rates for creating new cell lines from primary cultures, and define the minimal essential factors required for insect cell proliferation.

We found that the polyamines and spermidine in particular is important for enabling long-term maintenance of C1.8 cells in a chemically defined medium, and that Bruton's tyrosine kinase inhibition may also be an important target to stimulate proliferation in this CDM. All positive hit compounds can be important, and one or a combination of these compounds can be used for a media to support the long-term culture of the S2 cell line, as well as other insect cell lines.

### Example 2

### An Inverse Small Molecule Screen to Design a Chemically Defined Medium Supporting Long-Term Growth of Drosophila Cell Lines

To characterize the minimal requirements for long-term maintenance of Drosophila cell lines, we developed an inverse screening strategy to identify small molecules and synergies stimulating proliferation in a chemically defined medium. In this chemical-genetics approach, a compound-protein interaction database is used to systematically score genetic targets on a screen-wide scale to extract further information about cell growth.

In the pilot screen, we focused on two well-characterized cell lines, Clone 8 (C1.8) and Schneider 2 (S2). Validated factors were investigated for their ability to maintain cell growth over multiple passages in the chemically defined media (CDM) described herein. The CDM described herein supports the long-term growth of both C1.8 and Kc167 cells. Iterations of the screen can enable the identification of compound combinations optimized for specific applications—maintenance and generation of new cell lines or the production and purification of recombinant proteins—thus increasing the versatility of Drosophila cell culture as both a genetic and biochemical model system.

Materials and Methods.

Cell Culture.

C1.8, S2, and Kc167 (Kc) cells were expanded in optimized serum-containing media, C1.8, S2, and Kc167 media, respectively. C1.8 media contains M3 media (Sigma-Aldrich) supplemented with FBS (2%), insulin (5 μg/m), and fly extract (2.5%). S2 media is based on Schneider's media (Gibco® Life Technologies) supplemented with FBS (10%). Kc media containing M3 media supplemented with yeast extract (10 mg/mL), bactopeptone (25 mg/mL), and 5% FBS. All cell lines were obtained and cultured according to instructions from the Drosophila Genomics Resource Center (DGRC). Fly extract was prepared from adult yw flies as described by the DGRC. For culture in chemically defined media, cells were rinsed three times in PBS to remove residual serum and seeded at around 70% confluency in ZO Fortified or ZB Media (ZO Fortified with 1 μM spermidine added at time of passage). Upon reaching confluency, cells were passaged 1:2 retaining half the spent media, similar to routine maintenance in complete media, with cells passaged in ZB Media receiving fresh doses of 1 μM spermidine.

Basal Media.

While Wyss' ZO medium was never widely adopted, we selected it as the starting point for designing a completely chemically defined basal medium as it represents the most recent and thorough systematic effort to create a chemically defined medium for Drosophila. ZO media was initially acquired commercially (Sweden National Veterinary Institute) but is currently prepared in our lab in small batches as described by Wyss with similar results (Wyss, Exp. Cell Res., 1982, 139, 297-307; Wyss, Exp. Cell Res., 1982, 139, 309-319).

Preliminary efforts focused on testing compound candidates from the literature for their effect on proliferation. Various proliferation assays were tested, with CyQUANT® Direct Proliferation Assay (Life Technologies) yielding the best calibration between fluorescent intensity and cell number. CyQUANT is a DNA content-based assay that uses a background suppressing dye that is selectively permeable to dead cells (lacking membrane integrity), enabling specific labeling of live cells (Zartman et al., Dev. Camb. Engl., 2013, 140, 667-674). Conveniently, this assay yields good calibrations between cell number and fluorescent intensity (r²>0.95) even when used at 0.25× the suggested working concentration.

Using this approach we developed an intermediate “ZO Fortified” medium, made up of ZO media supplemented with insulin (5 μg/mL), trehalose (26.4 mM), L-alanyl-L-glutamine (ala-gln, 12 μM), and L-ascorbic acid 2-phosphate sesquimagnesium salt hydrate (A2P, 0.08 μM), with pH adjusted to 6.75. Insulin is a growth stimulator through insulin receptor signaling; trehalose is a disaccharide present in insect hemolymph; ala-gln is a stabilized dipeptide version of the essential amino acid L-glutamine; A2P is a stable form of L-ascorbic acid. Whereas ZO unsupplemented does not support proliferation of C1.8 cells, ZO Fortified supports short-term cell growth and initial attachment (FIG. 14C). ZO Fortified is capable of supporting slow growth of C1.8 cells through approximately 3 passages.

Pilot Screen.

To more efficiently identify compounds important for cell growth in serum-free media, we selected five small molecule libraries for pilot screening: the Wnt Pathway Library (75 compounds), Autophagy Library (97 compounds), Kinase Inhibitor Library (80 compounds), Phosphatase Inhibitor Library (33 compounds), and Ion Channel Ligand Library (72 compounds) (Enzo® Life Sciences). The Wnt pathway is important for regulating cell proliferation and differentiation. The Autophagy Library encompasses the target of rapamycin (TOR) pathway, involved in the regulation of growth and apoptosis with respect to nutrition, and is important in cancer. The Phosphatase and Kinase Libraries target tumor suppressors and oncogenes involved in TOR signaling, as well as other kinases and phosphatases involved in cell growth, proliferation, and survival. Ion channels have well-known roles in cell proliferation and cancer. These libraries come in a convenient 96-well format in dimethyl sulfoxide (DMSO) at a concentration of 10 μM.

Because most compound targets are identified using mammalian models, their efficacy and specificity to Drosophila are largely unknown. Further, many of the compounds tested are likely growth inhibitors rather than stimulators. To supplement the commercial libraries, a custom library was created that includes supplements that could be important for cell growth (39 unique compounds/supplements, 53 total; Table A, Example 1, above).

Preliminary investigations yielded working concentrations for some of these compounds; therefore, custom library stocks were prepared either at 10 μM (for un-tested compounds) or 10,000× their working concentration (based on preliminary tests). The vast majority of these compounds stocks were prepared in DMSO, but some were prepared in water or ethanol based on solubility (see Table A, Example 1). Based on the screening concentrations selected, the final DMSO dilution is 1:1000 or greater, and thus not expected to impact results. These 6 libraries served as the basis for the pilot screen. The combined contents of the tested libraries along with raw scores from the screening experiments are listed in the electronic supplementary information of Burnette et al., Mol. BioSyst., 2014, 10, 2713-2723, which documents are incorporated herein by reference

Five stock plates of randomized compounds in singlet (410 total) were prepared at 75 μM in water (1:133 dilution) using an automated liquid handler (Eppendorf epMotion® 5075). Ten strategically located negative controls were incorporated into each stock plate by dosing wells with DMSO (1:133, carrier only control) (Zhang, Optimal High-Throughput Screening: Practical Experimental Design and Data Analysis for Genome Scale RNAi Research, 2011). Four randomly placed positive control wells were also included on each plate by dosing wells with DMSO (1:133) for consistency, but for flexibility positive control treatments were prepared in the working stock. Working stocks at 2 and 20 μM (2×) were then prepared in ZO Fortified by diluting the 5 stock plates 1:3.75 (high concentration working stock) and then serially diluting 1:10 (low concentration working stock). Positive control wells were prepared by replacing 10% of the media with 100% fly extract (FEX) (see Zartman et al., Dev. Camb. Engl., 2013, 140, 667-674).

Cells were rinsed three times in phosphate buffered saline (PBS) and seeded at 50,000 cells/well (50 μL) in ZO Fortified. Working stocks, 50 μL, were then immediately added on top of the cells (day 0). Plates were incubated at 25° C. for four days, and then imaged at 10× magnification on a Nikon microscope using automated screen acquisition in MetaMorph® software. Plates were then treated with CyQUANT® (0.25× final concentration) for one hour at 25° C., and fluorescent intensity was measured with a plate reader (Biotek Synergy H2).

Screen Analysis and Hit Selection.

Strictly standardized mean difference (SSMD) scores for classifying assay quality were calculated with respect to negative controls on a plate-wise basis, with the average assay quality being excellent for both cell lines and screened concentrations. We investigated the effect of performing positional corrections (median polishing, etc.) to account for plate-location effects and found that screen quality, as assessed by the SSMD, were overall best when no positional corrections were made. As a hit selection metric we used z-scores, which measure the number of standard deviations from the negative control that a small molecule treatment causes. Z-scores were calculated on a plate-wise basis, and hit cutoffs of z=±3 were selected. Visual inspection of images was used to remove false-positive hits, which generally occurred due to autofluorescence of a few of the compounds.

Growth Kinetics Experiments.

Cells were rinsed three times in PBS and seeded in 96-well plates at a concentration of 50,000 cell/well (by plating 5 μL of a 1×10⁷ cell/mL suspension in PBS on top of 95 μL of media). Three replicates per media were assayed with CyQUANT (0.25× final concentration) daily, starting immediately after seeding (day 0). After addition of CyQUANT, cells were incubated for one hour at 25° C., and fluorescent intensity was measured with a plate reader (Biotek Synergy H2) at a gain of 70 to facilitate day-by-day comparisons in intensity. A fold change in intensity of >1 indicates proliferation of cells.

Polyamine-Depleted Spermidine Dose-Response Measurements.

C1.8 cells were rinsed three times in PBS and seeded in ZO Fortified at 1×10⁶ cell/mL. After nine days of culture in ZO Fortified, cells were harvested and seeded in tissue culture flasks in ZO Fortified supplemented with 0, 0.1, 1, or 10 μM spermidine (in triplicate). After 6 days of culture, cells were manually counted.

Cell Immunostaining and Western Blots.

For EdU incorporation, C1.8 and S2 cells were rinsed three times in PBS and seeded in optical grade 96-well plates at a concentration of 50,000 cell/well (by plating 5 μL of a 1×10⁷ cell/mL suspension in PBS on top of 95 μL of media). After four days of culture, cells were assayed with Invitrogen's Click-iT® EdU Alexa Fluor® 647 Imaging Kit according to their instructions, with a three hour EdU incorporation time. Cells were then stained with DAPI (1:1000) and two positions per well were imaged at 40× magnification on an EVOS® fluorescent microscope (AMG). CellProfiler, a customizable image analysis package was used to quantify total number of cells (from DAPI images) and number of EdU positive cells (from channel 647 images) within each image (see Carpenter et al., Genome Biol., 2006, 7, R100). A minimum of 375 cells was analyzed for each condition tested.

For western blots, C1.8 and S2 cells were rinsed three times in PBS and seeded in tissue culture flasks at a concentration of 1,000,000 cell/mL in the various media. Cells were cultured until positive control samples (complete media) reached ˜90% confluency, at which point all cell samples were lysed for 30 minutes on ice in a buffer containing 50 mM Tris, 150 mM NaCl, 1% Nonidet P 40 substitute (Sigma 74385), and 1% Sigma protease inhibitor cocktail (P8340). Nucleic acid and cell debris were cleared by centrifugation at 12,000 rpm for 20 minutes at 4° C. Because polyamines are known to play roles in initiation of transcription and translation, “housekeeping” genes routinely used as loading controls for western blots are likely inappropriate for this application. Thus samples were normalized by total loaded protein. Sample protein concentrations were determined three independent times per sample using Coomassie Plus (Bradford) Assay (Thermo Scientific 23238). ANOVA comparison of loading concentrations across all samples found no significant variation between samples (p>0.34). Proteins were resolved by SDS-PAGE in reducing conditions, electroblotted to PVDF membranes (Hybone 10600087), and probed with either 1:1000 anti-rabbit p44/p42 MAPK (Erk1/2) (Cell Signaling) or 1:1500 anti-mouse MAPK (dpERK1/2) (Sigma). Blots were developed using WesternBreeze chromogenic western blot immunodetection kit (Invitrogen) and quantification was done in Fiji (Schindelin et al., Nat. Methods, 2012, 9, 676-682).

Compound Target Analysis.

The Search Tool for Interactions of Chemicals (STITCH) database was used to identify protein targets of the tested compounds (see Kuhn et al., Nucleic Acids Res., 2010, 38, D552-D556). STITCH integrates data from multiple databases and catalogs the interactions between more than 300,000 compounds and 2.6 million proteins in 1,133 different species (Kuhn et al., Nucleic Acids Res., 2011, 40, D876-D880). The STITCH download file of chemical-protein links was queried for all of the screened compounds, and interactions and combined interaction scores for human (Homo sapiens), mouse (Mus musculus), and Drosophila melanogaster were compiled, resulting in a list of 23,158 uniquely targeted proteins. To assign one score per interaction and penalize inconsistent results, z-scores at high and low concentration were averaged. Drugs yielding no STITCH results were removed from the analysis (78 compounds total). Drosophila orthologs and % identity match scores for the human and mouse proteins were then found using Ensembl (Flicek et al., Nucleic Acids Res., 2012, 41, D48-D55). Proteins yielding no orthologs were removed from analysis (9,491 unique proteins).

This analysis resulted in a list of 6,090 proteins (˜22% of the Drosophila protein-coding genome) that were potentially targeted by the small molecule screen. In order to “translate” the compound scores to a scored protein list, we began by linking the absolute value of each average z-score to each compound-protein interaction. The average of the two concentration z-scores was used to penalize compounds that yielded inconsistent results. Because the database (STITCH) is rooted in text-mining, interaction scores do not provide any information about whether a given compound acts as an inhibitor or activator of its target protein. Thus absolute values were used. To generate protein lists of targets important for stimulating versus inhibiting proliferation, compounds were separated into potential proliferation agonists or antagonists (average z-score positive or negative, respectively), and targets were scored for these two sets of compounds separately.

Specifically, for each n compound related to d Drosophila proteins, each i^{th} relation has an interaction score (sᵢ) and % identity match score (qᵢ) between 0 and 1, and z-score zᵢ. Approximately then, the protein score, Σ(sᵢ·qᵢ·zᵢ²), follows a chi-squared distribution with degrees of freedom n, and p-values can be calculated accordingly. These protein p-values are thus based on: 1) the strength with which their interactor compounds affected proliferation (absolute z-score), 2) the confidence level of the compound-protein interaction (sᵢ), 3) the number of times a protein was targeted by unique compounds (summing scores per targeted protein), 4) the degree of conservation between originally targeted mammalian proteins and their Drosophila orthologs (qᵢ).

To generate significantly targeted protein lists and account for multiple hypothesis testing, we used the standard Bonferroni correction and implemented a p-value cutoff of (0.05/d) for each set of scores. Drosophila protein IDs were converted to gene IDs using FlyBase (St. Pierre et al., Nucleic Acids Res., 2013, 42, D780-D788). We then used the Database for Annotation, Visualization, and Integrated Discover (DAVID) to identify enriched Gene Ontology and KEGG annotations (see Huang et al., Nucleic Acids Res., 2009, 37, 1-13; Huang et al., Nat. Protoc., 2009, 4, 44-57). Importantly, because our library does not target the entire proteome, this resource enables definition of the background facilitating accurate determination of pathway enrichment. Finally, REVIGO was used to reduce redundancy in ontology annotations by grouping them into terms, which are visualized in semantic similarity plots (Supek et al., PLoS ONE, 2011, 6, e21800).

Results.

Small Molecule Screen in ZO Fortified.

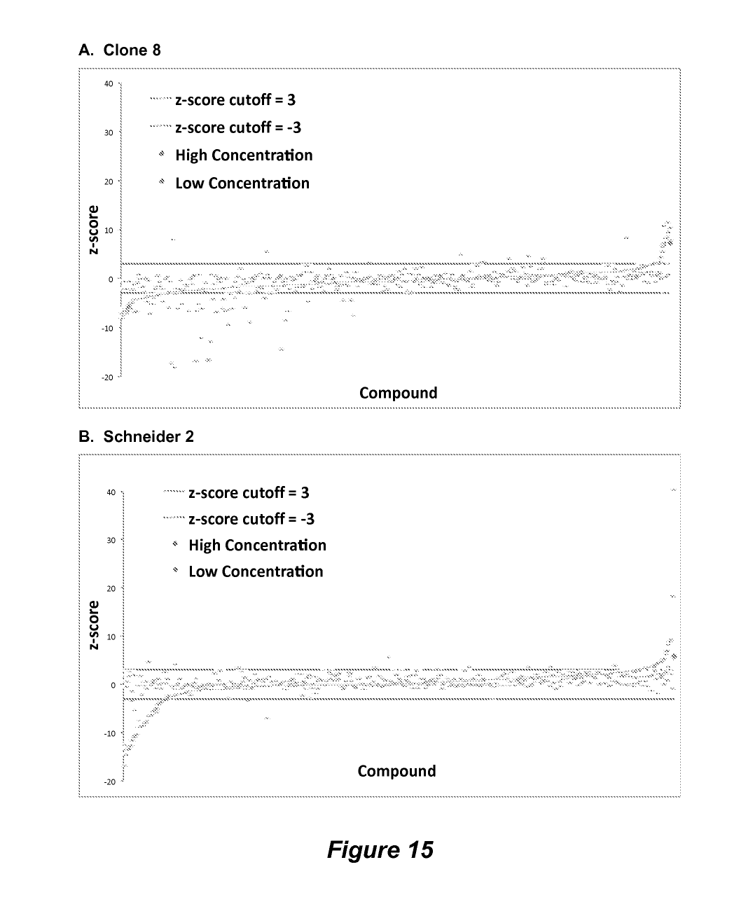

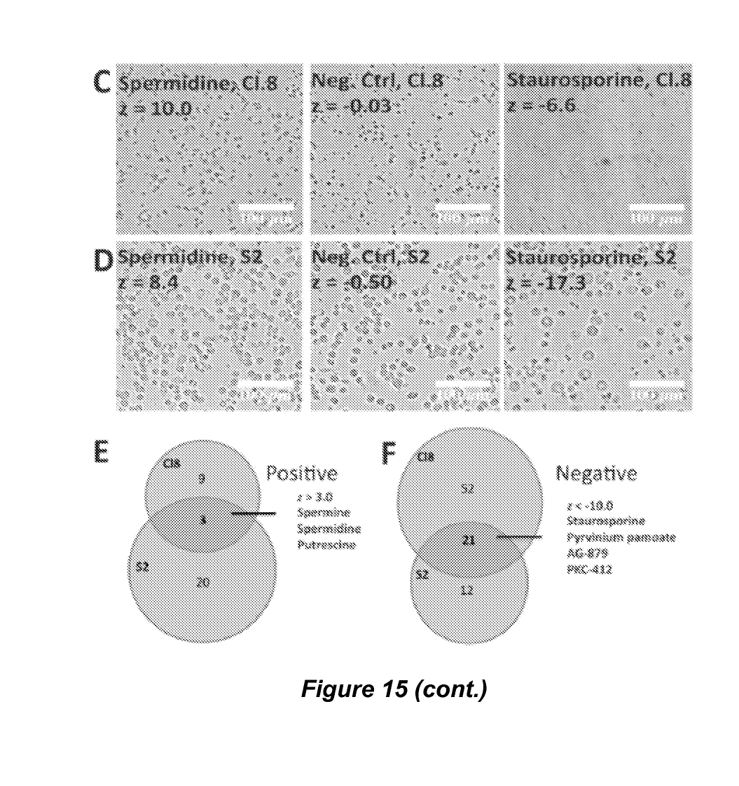

From the screen, 12 and 20 unique positive hit compounds that increase cell numbers were identified for the C1.8 and S2 cell lines, respectively, and 73 (for C1.8) and 33 (for S2) unique negative hit compounds decreased cell numbers (FIGS. 3C, 4,15E (positive hits), 5, 15F (negative hits)). Of the positive hits, three compounds were identified in both cell lines. All of the repeated positive hits were polyamines (spermine, spermidine, and putrescine), with spermine (z_{C1.8}=10.4, z_{S2}=17.7) and spermidine (z_{C1.8}=10.0, z_{S2}=8.7) representing the two strongest positive hits in both cell lines (FIG. 15E).

Of the negative hits, 21 compounds were found in both cell lines (FIG. 15A, F). To narrow the analysis, another threshold was applied, yielding 4 compounds with z-scores<−10 in both cell lines: pyrvinium pamoate (z_{C1.8}=−17.7, z_{S2}=−13.0), staurosporine (z_{C1.8}=−18.6, z_{S2}=−17.3), AG-879 (z_{C1.8}=−17.3, z_{S2}=−13.9), and PKC-412 (z_{C1.8}=−17.1, z_{S2}=−11.0) (FIG. 15F). Pyrvinium pamoate is an androgen receptor inhibitor and anticancer agent. Staurosporine and PKC-412 are inhibitors of protein kinases, with staurosporine being the precursor for PKC-412 development. AG-879 is tyrosine kinase inhibitor and suppressor of malignant transformation. Staurosporine is a known autophagy inducer, indicating that our approach does detect cell number changes resulting from compound treatments.

Growth Properties in Chemically Defined Medium.

Due to the lower cost of spermidine and our desire for an economical CDM we incorporated 1 μM spermidine into ZO Fortified, newly termed “ZB Media,” and conducted growth kinetics experiments to characterize the effect of supplementing ZO Fortified with spermidine. To determine the extensibility of this medium, the growth of Kc167 cells, an isolate of the Kc167 embryonic cell line, was also investigated.

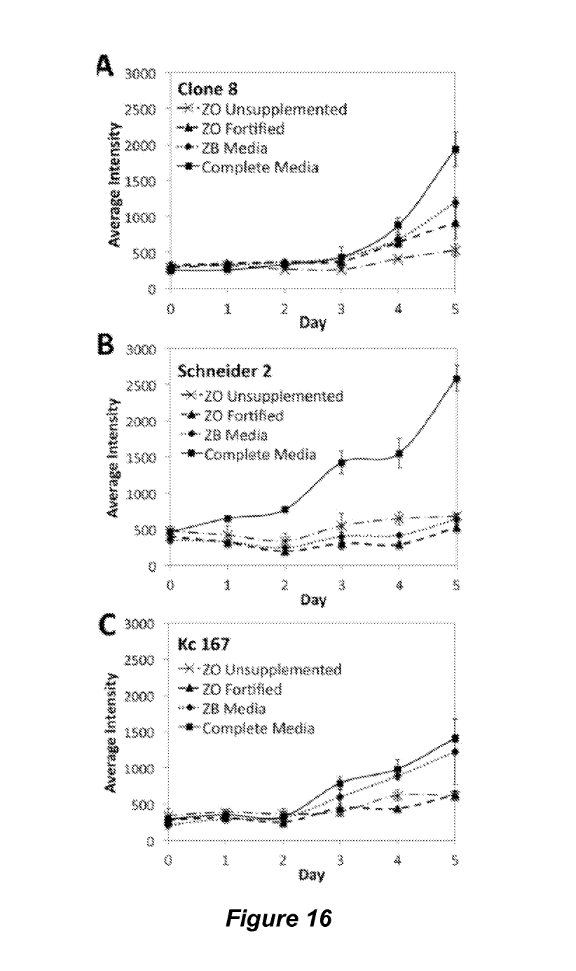

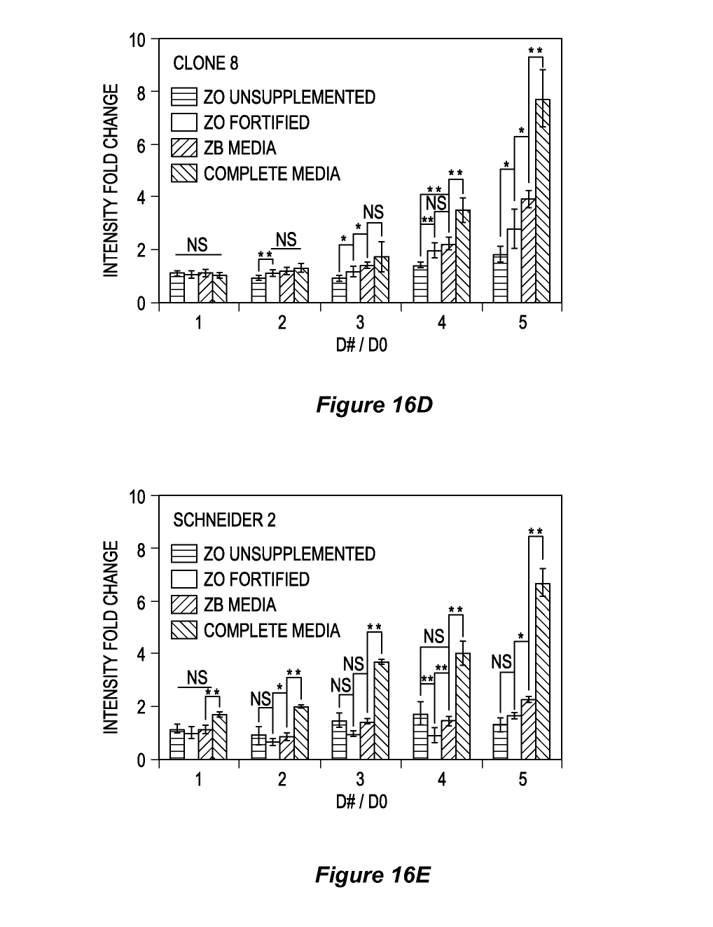

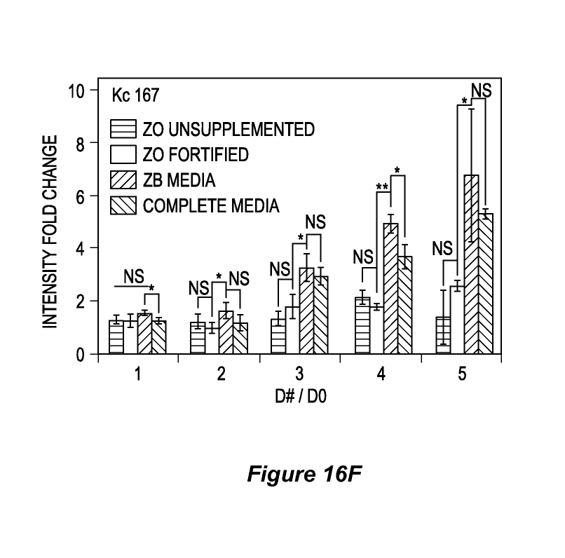

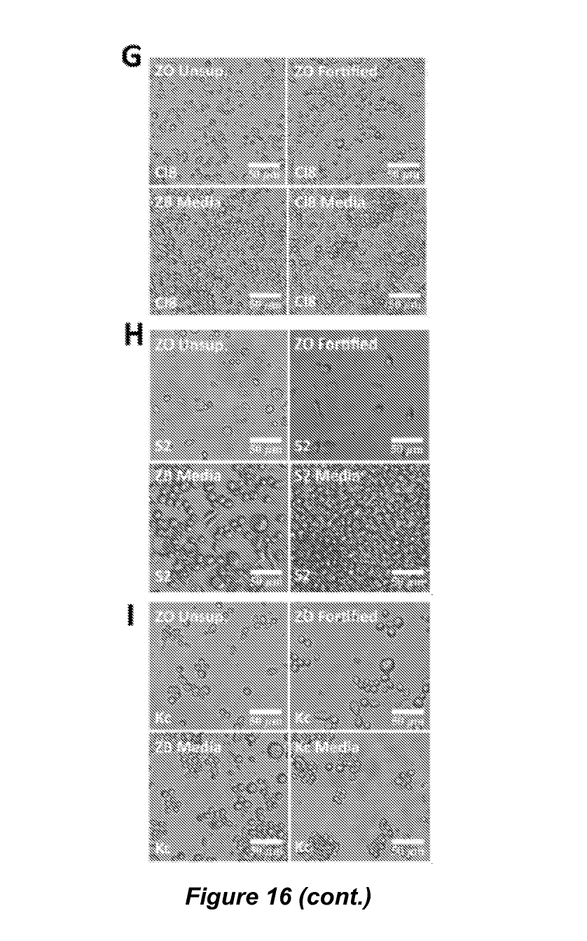

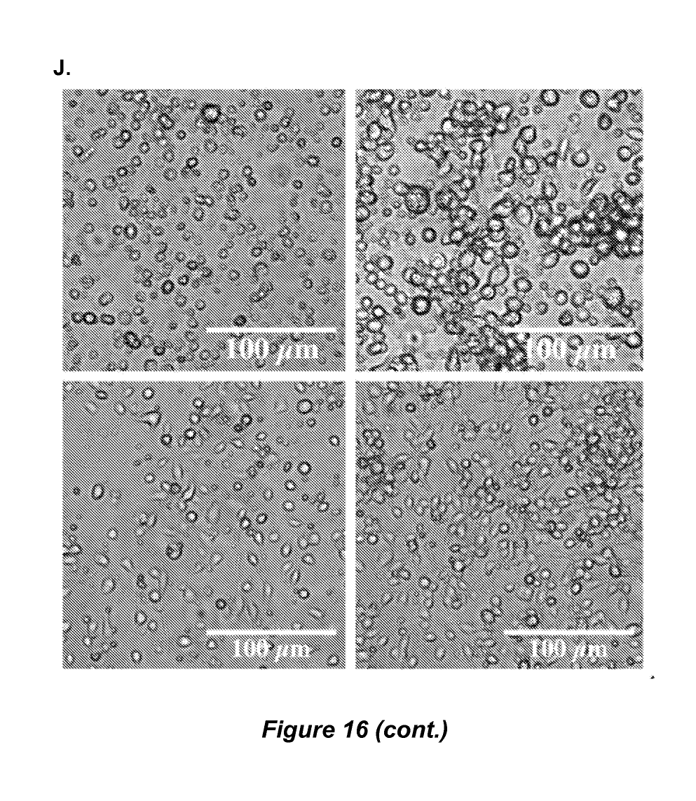

For all three cell lines tested, ZB Media yields higher proliferation rates than ZO Fortified, indicating that spermidine does indeed promote cell growth and attachment (FIG. 16A-C, G-I). Kc167 cells in particular proliferate in ZB Media at comparable rates to in the complete serum-containing medium. Interestingly, ZO Fortified, which we developed for C1.8 cells, does not significantly improve growth compared to ZO unsupplemented for S2 or Kc167 cells (FIG. 16D-F).

Long-Term Culture in Chemically Defined Medium.

To determine ZB Media's ability to support long-term cell growth, we cultured C1.8, S2, and Kc167 cells in ZB Media versus ZO Fortified. After 3 passages in the two chemically defined media, C1.8 cells continue to proliferate in ZB media (ZO Fortified supplemented with spermidine) whereas those cultured in ZO Fortified (no spermidine supplementation) stall in growth (FIG. 16J).

Inclusion of spermidine has enabled C1.8 growth through 98 passages (thus far) with no signs of decreasing growth rates. Importantly, cell morphology in ZB Media is consistent with the serum containing media. Surprisingly, C1.8 cells do not require a weaning from serum to adapt to the CDM; they adapt quickly to sustained growth in ZB Media. Adapted C1.8 cells (passaged 10 times in ZB Media) passaged 1:2 become confluent after 1-2 days, comparable to C1.8 cells cultured in C1.8 medium. Adapted C1.8 cells can also be passaged at higher dilutions (1:6) and reach confluency within 3-4 days. Further, an investigation into the growth kinetics of C1.8 cells at various seeding densities shows that spermidine improves growth of cells in ZO Fortified even at low concentrations, and that C1.8 cells proliferate in ZB Media even when seeded at low concentrations. To determine if there is any dependence of proliferation on potential secreted factors, we have performed spent media titrations, which fail to show any benefit on growth induced by spent media. ZB media is also capable of supporting growth of Kc167 cells through at least 14 passages without any signs of stalling in growth.

S2 cells grown in ZB Media reach passage 4 before growth stalls whereas those grown in ZO Fortified are unable to progress through passage 1 (FIG. 16J). Thus while ZB Media enables additional growth of S2-DSRC cells, ZB Media requires additional growth-activating compounds to support long term growth of S2 cells.

Therefore, we have developed a medium capable of supporting long-term growth of C1.8 and Kc167 cells by identifying a supplement cocktail of 5 components. C1.8 cells adapted to ZB media are able to recover after storage in liquid nitrogen in ZB Media supplemented with 0.2 M trehalose and 10% DMSO. We have found that inclusion of trehalose in freezing media significantly improves cryopreservation in multiple media/cell types, consistent with findings for mammalian cells.

Biological Effect of Spermidine.

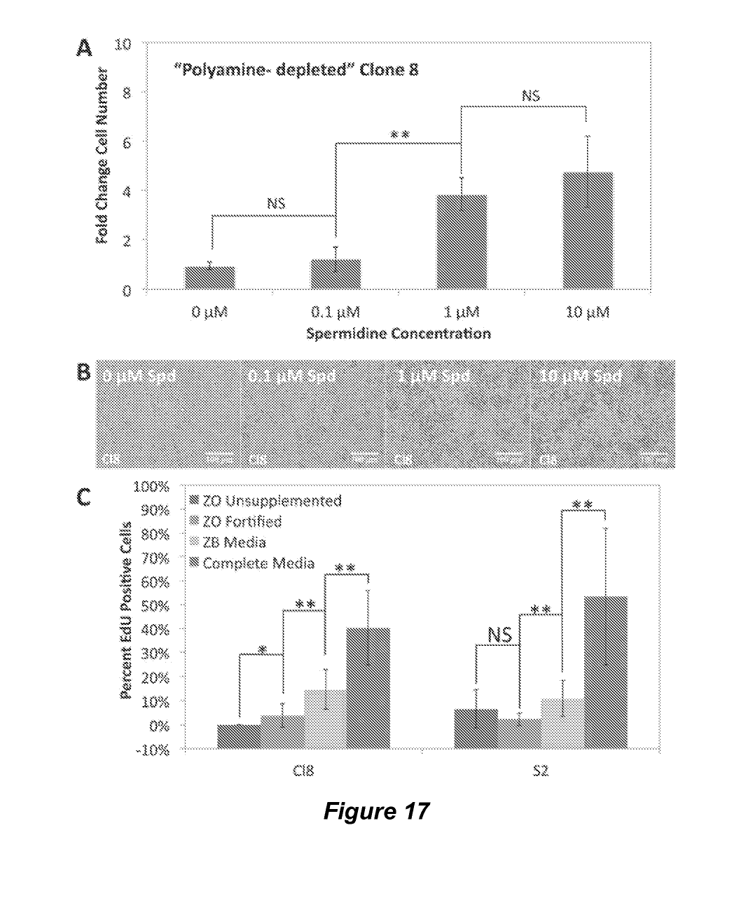

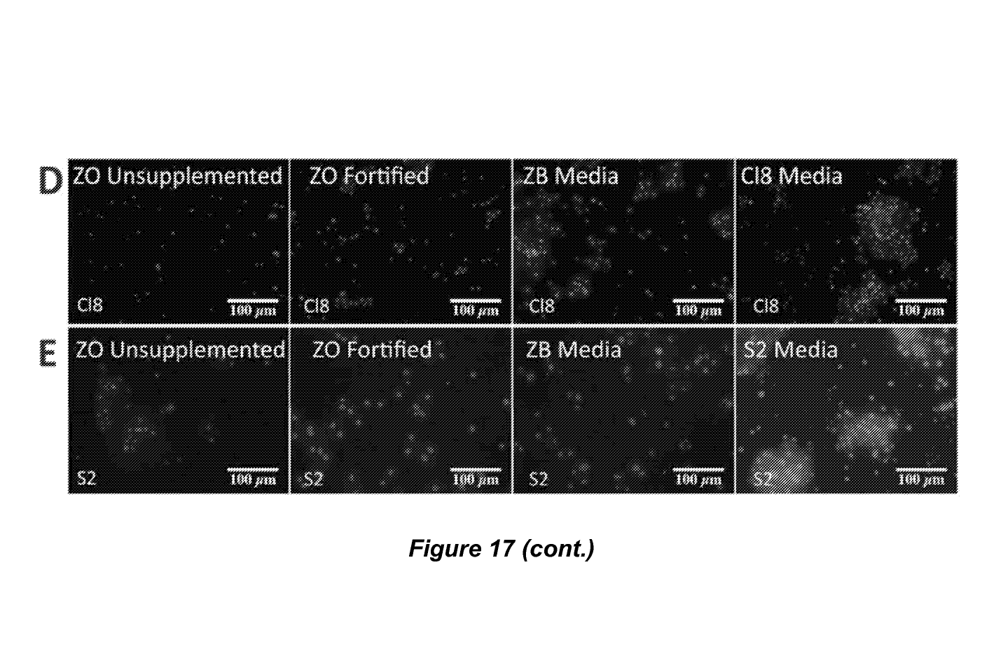

Short-term dose-response experiments failed to show strong dose-dependence of proliferation on spermidine concentration. However, when polyamine-depleted C1.8 cells were used, a clear dependence of proliferation on spermidine concentration was observed (FIG. 17A-B). Supplementation with 1 μM spermidine caused a significant increase in proliferation (p<5×10⁻¹⁰) of C1.8 cells compared to 0 or 0.1 μM spermidine supplementation. A higher dose, however, failed to produce any statistically significant increases in cell number after 6 days.

To more specifically show that spermidine does indeed promote proliferation, we measured DNA synthesis by 5-ethynyl-2′-deoxyuridine (EdU) incorporation experiments on C1.8 and S2 cells. ZB Media yields a significantly higher percentage of EdU positive cells than ZO Fortified for both C1.8 (p<0.005) and S2 (p<0.025) cells (FIG. 17C-E).

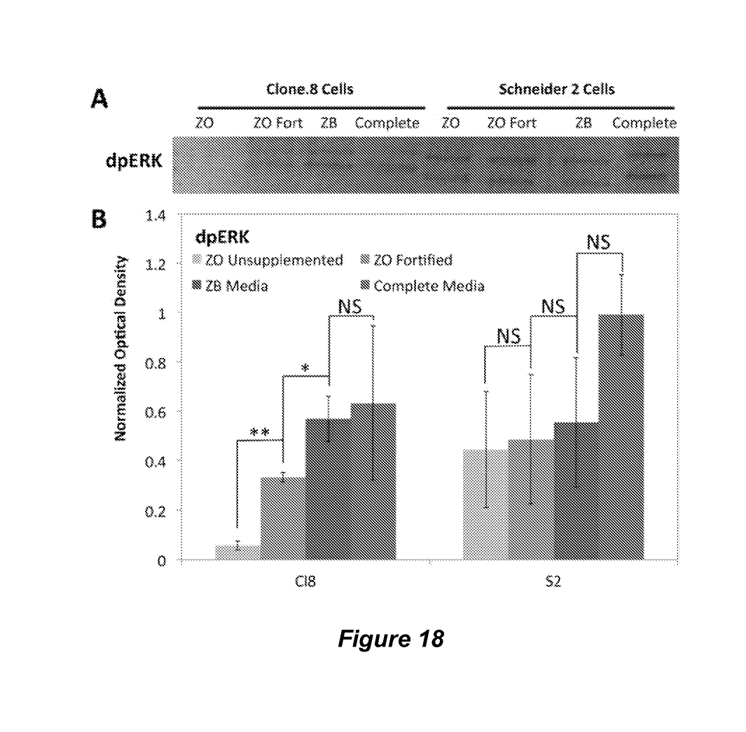

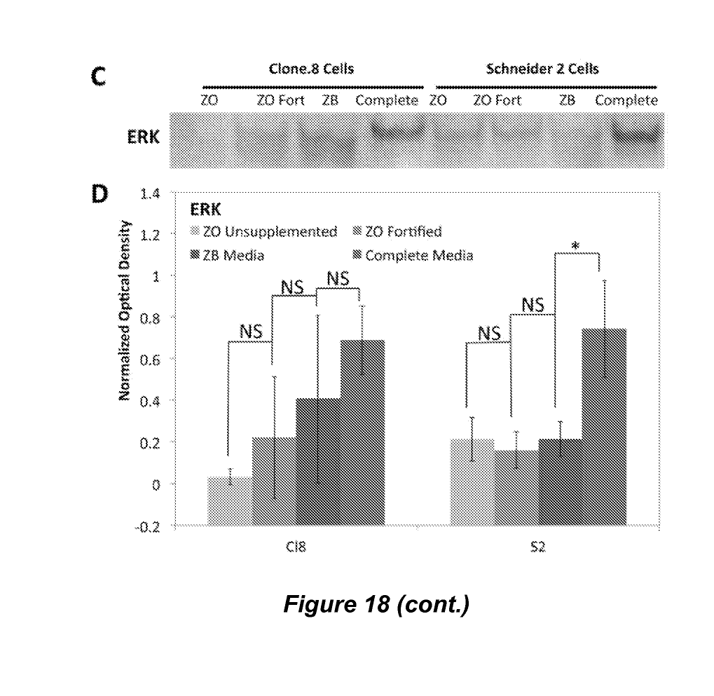

Because spermidine can stimulate phosphorylation of tyrosine kinases and ERK1/2 in the Ras/MAPK signaling cascade, we also investigated MAPK activity of C1.8 and S2 cells in our spermidine supplemented medium. Western blots indicate that spermidine supplementation does in fact increase ERK double phosphorylation for C1.8 cells (p<0.012), but not for S2 cells (p>0.75) (FIG. 18A-B). Total ERK levels are not significantly influenced by spermidine supplementation for either cell line (p>0.45), although there is a trend of increasing ERK levels across the iterating improvements to Wyss' original ZO Media (FIG. 18C-D).

Compound Target Analysis.

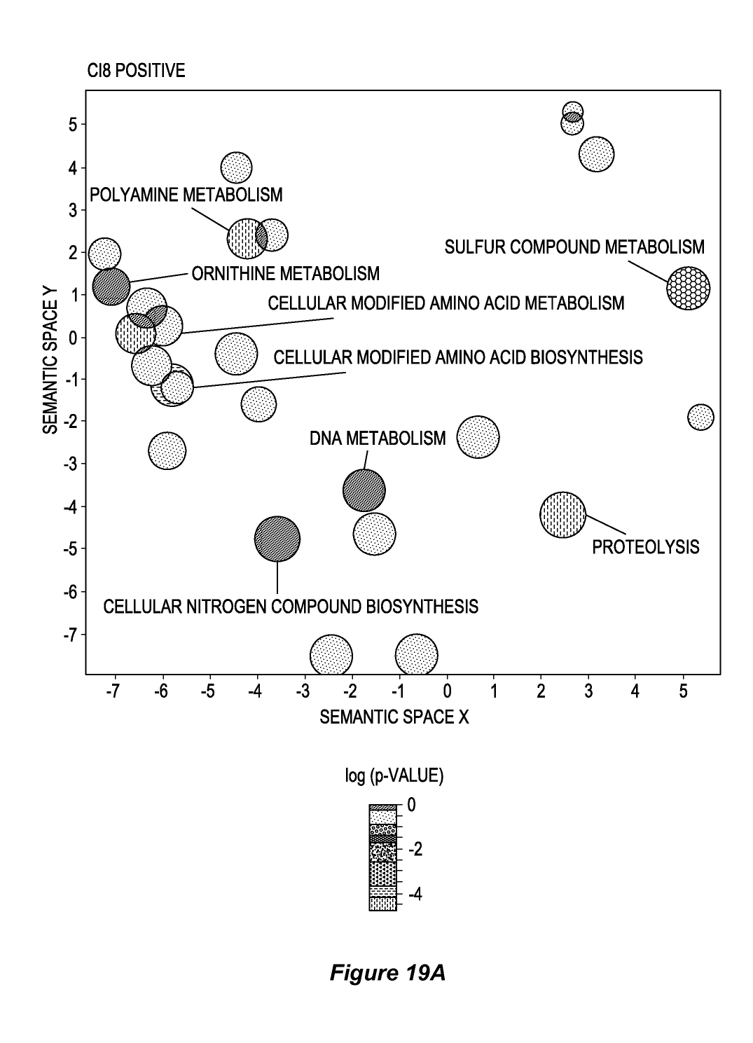

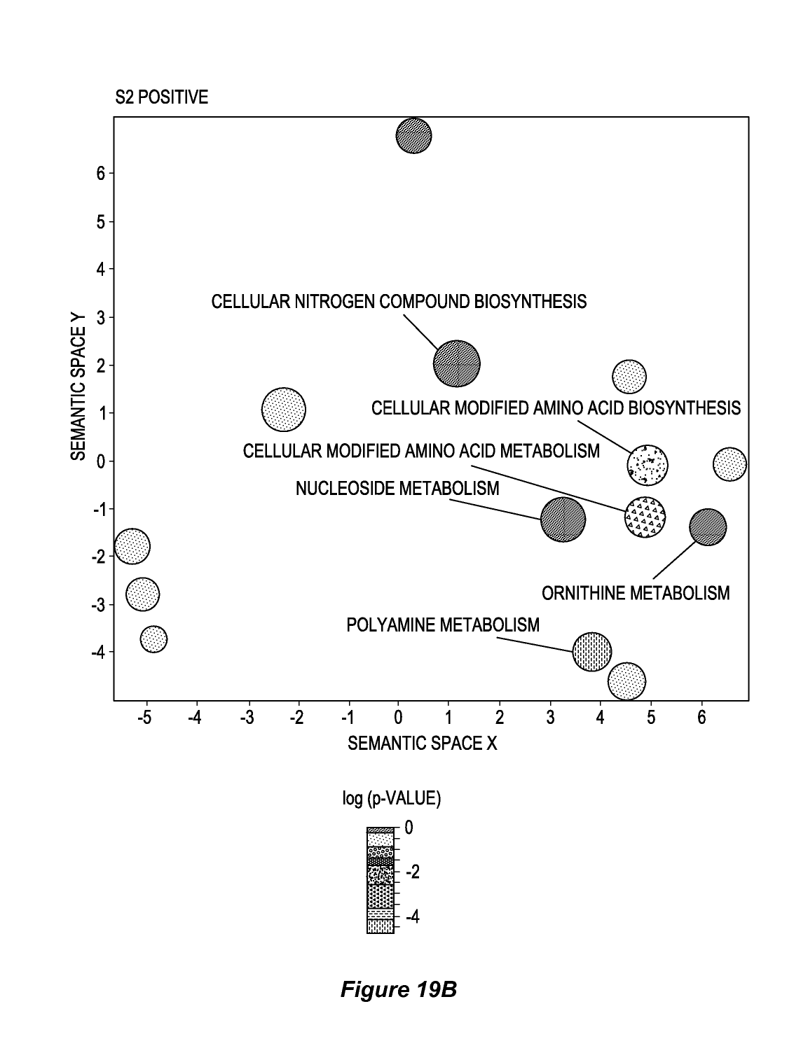

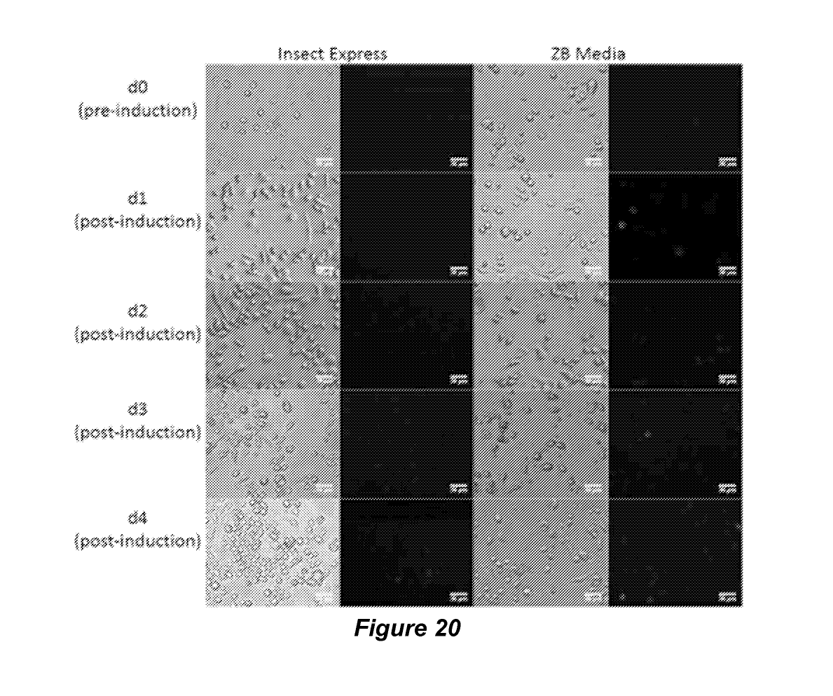

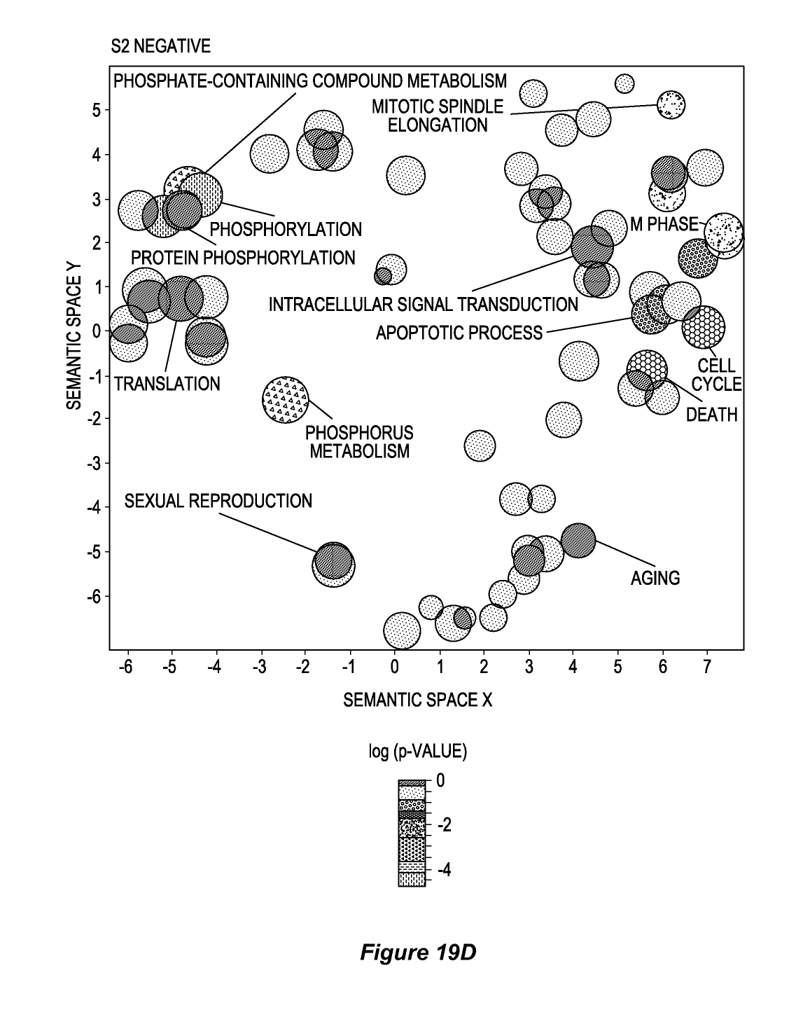

Target analysis on compounds with positive average z-scores yielded 111 gene product candidates significantly targeted for the C1.8 cell line and 53 gene product candidates significantly targeted for the S2 cell line. All of the gene products that were targeted in S2 were also targeted in C1.8. Some of the strongest genes targeted by positive-scoring compounds for both cell lines were glycogen phosphorylase (GlyP), ornithine decarboxylase 1 (ODC1), S-adenosylmethionine decarboxylase (SamDC), casein kinase IIα (CkIIα), and ornithine aminotransferase precursor (Oat). These findings are consistent with the importance of polyamines, with ODC1 and SamDC both being central upstream enzymes in the polyamine biosynthesis pathway. Ontology enrichment on scored protein lists yielded multiple annotations that were visualized using REVIGO scatter plots, which group similar annotations into broader “terms” and plot them on semantic axis where similar terms are closer together; points on scatter plots are sized according to the number of ontology annotations per term and are colored based on their p-values (FIG. 19). Among enriched ontology terms for targets of positive z-score compounds, polyamine metabolism, ornithine metabolism, and cellular modified amino acid biosynthesis pathways emerged for both cell lines.

KEGG pathway enrichment was also conducted for targets of positive-scoring compounds, with glutathione metabolism, arginine and proline metabolism, and cysteine and methionine metabolism all significantly enriched terms for C1.8 cells, and arginine/proline metabolism and cysteine/methionine metabolism being significantly enriched for S2 cells as well. This is consistent with the importance of polyamine metabolism for cells cultured in ZO Fortified, as polyamines are synthesized from both arginine and methionine. The first step in polyamine metabolism is the production of ornithine from arginine; at the same time, L-methionine is used to create decarboxylated S-adenosyl-L-methionine (DcAdoMet), which acts as an aminopropyl group donor to either putrescine or spermidine to produce either spermidine or spermine, respectively.

Target analysis on compounds with negative average z-scores yielded 266 gene product candidates significantly targeted for the C1.8 cell line and 166 gene product candidates significantly targeted for S2 cell line. Some of the strongest genes targeted by negative-scoring compounds in both cell lines were phosphorylase kinase γ (PhKγ), calmodulin (Cam), and downstream of raf1 (Dsor1). Among enriched ontology terms, protein amino acid phosphorylation, protein kinase activity, phosphorus metabolic process, and ATP binding emerged. The target of rapamycin (TOR) pathway and progesterone-mediated oocyte maturation were found as significantly enriched KEGG pathways for C1.8 cells, with ribosome as well as again the progesterone-mediated oocyte maturation being enriched for S2 cells. The TOR pathway is strongly involved in the regulation of cell growth, proliferation, and survival, especially in the context of coupling growth with nutrition.

A complete list of compounds, average z-scores, target proteins, as well as lists of target proteins and their associated p-values and enriched ontology annotations are provided in the electronic supplementary information of Burnette et al., Mol. BioSyst., 2014, 10, 2713-2723, which are incorporated herein by reference. Validation of candidates and whether the targeting is definitively antagonistic or agonistic can be determined using standard methods known to those of skill in the art.

Discussion.

In this study, we have identified five compound additives that enable Wyss' ZO medium to support long-term growth and maintenance of Drosophila C1.8 and Kc167 cells. In particular, the polyamine spermidine was found through an inverse drug screen to be the critical component missing from ZO Fortified, our supplemented version of ZO medium that enables this long-term growth. The other two central polyamines, spermine and putrescine, were also identified from our compound screen to be significant growth promoters for both C1.8 and S2 cell lines. While many of the supplements used in culture media are undefined, it is known that polyamines are typically present in high levels in fermented foods such as yeast extract, which is routinely included in many Drosophila culture media. Drosophila cells may be missing upstream components for polyamine synthesis, or lack the signals to synthesize polyamines, and thus required exogenous polyamine supplementation.

Although polyamines aid proliferation, the exact biological functionalities for these molecules are both pleiotropic and incompletely defined. Here we show that spermidine supplementation significantly increases phosphorylation levels and expression levels of ERK. This is consistent with findings that spermidine specifically stimulates the phosphorylation of tyrosine kinases and ERK1/2 in the Ras/MAPK activated signaling cascade.

Importantly, the magnitude of the effects of spermidine on proliferation is relatively small, at least as measured in assays of short duration (4 days or less), indicating that polyamines become limiting only after significant cellular depletion. This assertion is also supported by the finding that dose-dependence of proliferation on spermidine concentration is stronger for cells that have been depleted of polyamines compared to normal cells. It appears that Drosophila cells may be unable to synthesize sufficient polyamines from the available nitrogen sources in ZO Fortified; therefore, polyamine supplementation is required and transport is likely crucial. This hypothesis is supported by the gene target analysis and ontology enrichment implicating the importance of arginine and methionine metabolism, both of which are required upstream for polyamine synthesis. In normal rat kidney cells, pharmacological knockout of polyamine synthesis required several days before polyamine levels were significantly reduced, potentially explaining why exogenous polyamine supplementation would have small effects in the short term but be required in the long term. Due to the suboptimal growth conditions of the screening media (ZO Fortified), use of two cell lines to increase screening resolution, and our protein-target analysis, we were able to detect the relatively small short-term effects of polyamine supplementation.

Our media development pipeline can be expanded for the rational design of media to improve conditions for industrial production of recombinant proteins from insect cells, increase success rates for creating new cell lines from primary cultures, or define the minimal essential factors required for the proliferation of other types of insect cells. However, one drawback of our pilot screen was the low genome coverage; STITCH target analysis indicated that only 22% of the proteome was targeted. This is due to our screening of a limited number of targeted compound libraries. This library selection and coverage issue is a promising application of our target analysis approach, which could be extended to develop rationally designed compound libraries that target the maximum possible genome coverage based on known interactions cataloged in STITCH. However, it is important to note that the target analysis approach is heavily dependent on the quality and quantity of data cataloged by interaction databases like STITCH.

One drawback of our method is the single-factor basis of our screening pipeline, which does not specifically identify compound synergies promoting proliferation. Pooling strategies specifically used for synergy identification, however, are not compatible with our target scoring approach⁸². An iterative expansion of the screen can be used to method to identify synergies, where validated factors can be incorporated into the media background before compound rescreening. This methodology can elucidate compound synergies with previously identified compounds, and our biological target identification technique coupled with this iterative approach can lead to important clues about novel biological mechanisms and crosstalk between pathways and proteins important for growth. Our cumulative gene target scoring approach is an improvement over traditional screens that only consider biological targets of individual hit compounds, and can be applied to query a wide range of biological processes in other model systems.

### Example 3

### Inducible Expression of Drosophila melanogaster GFP-Labeled Vein Variant in ZB Media

DNA Construct.

The cDNA of D. melanogaster vein was obtained from the DGRC. The DNA sequence was modified to remove the PEST sequence of the original protein in order to prevent or minimize proteolysis in this homologous expression system. A PEST sequence is a peptide sequence rich in proline (P), glutamic acid (E), serine (S) and threonine (T) that often mark proteins for degradation resulting in short intracellular half-life. The expression vector pMT-Bip-STABLE1-puro was a gift from Dr. J. D. Sutherland. This is a copper inducible insect cell expression vector that adds a signal peptide for secretion at the N terminus and a GFP module at the C terminus of the protein to facilitate microscopy imaging and fluorescence detection. PCR primers were designed to eliminate the PEST sequence and to create EcoR1 sites for cloning into the expression vector and to add the BiP signal peptide for protein secretion. The PCR forward primer was GTA GAA GCG AAT TCA ACA ACA TCG AC and the reverse primer was GGT TCG AAT TCA TGG GTT CC. The amplicon was digested with EcoRI and cloned into the EcoRI site of pMT-Bip-STABLE1-puro. Positive clones were screened by using EcoR1 and PstI. DNA sequencing of the construct was done at the Genomics and Bioinformatics Core Facility (Notre Dame) to verify the presence of signal peptide and the GFP tag to be in frame.

Transfection.

The D. melanogaster S2 cells were seeded at 8×10⁵ cells/mL in a 24 well plate format with a final volume of 360 μL. An amount of 0.2 μg of DNA per well was transfected using Effectene reagent following manufacturer's instructions (Qiagen). Expression of D. melanogaster PEST free vein was followed by GFP detection using Evos 20× and a Biotek plate reader. Stable polyclonal cells expressing the vein GFP (vn-GFP) fusion protein were generated by selection with puromycin for 10 days.

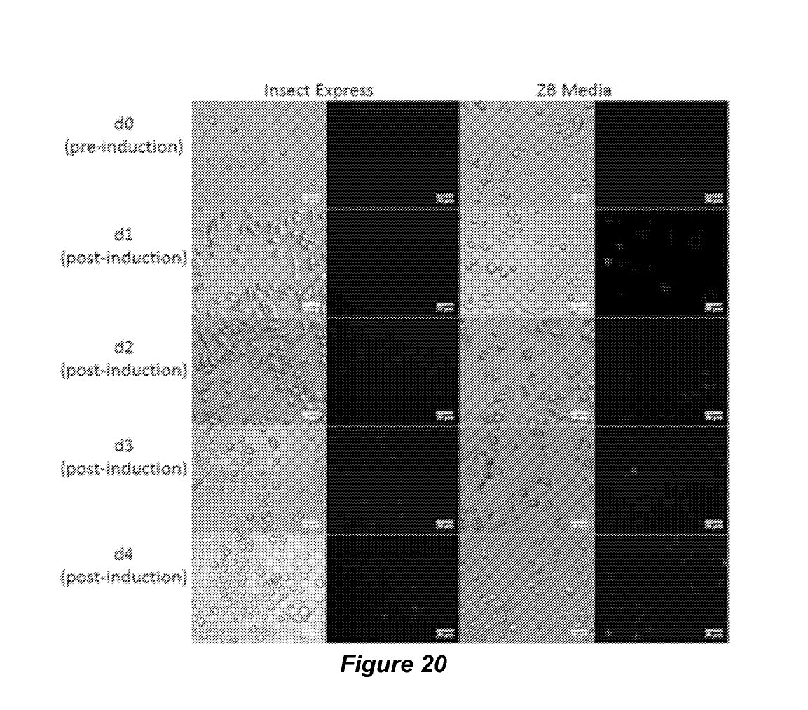

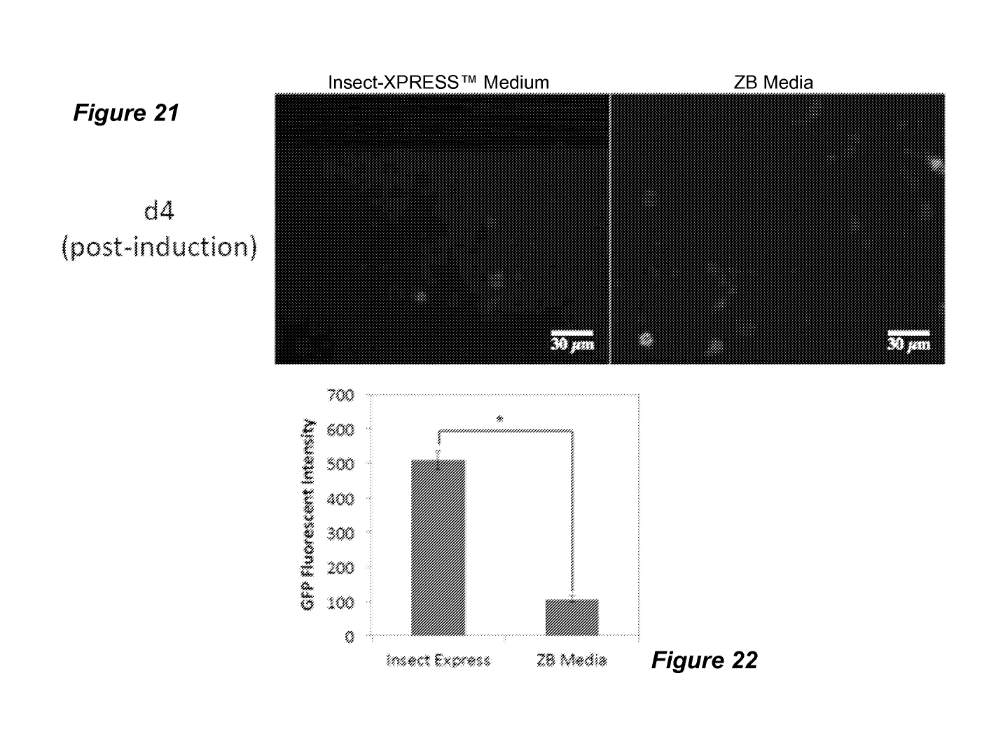

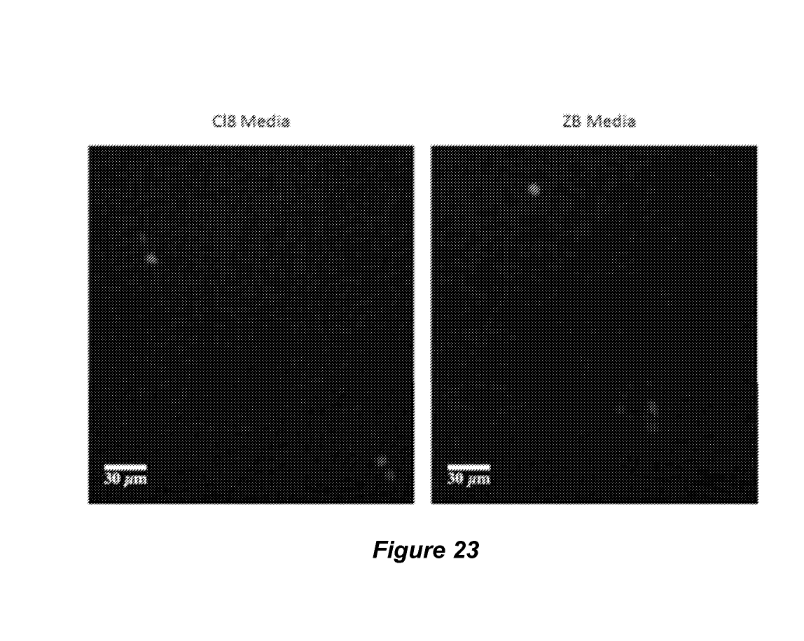

ZB Media yields higher recombinant protein expression than Insect-XPRESS™ Insect Cell Medium (Lonza) for S2 cells. FIG. 20 illustrates the higher recombinant protein expression in ZB Media compared to Insect-XPRESS™ Insect Cell Medium for S2 cells through four days. Stably transfected S2 cells with vn-GFP plasmid were rinsed three times in PBS and seeded at 500,000 cell/mL in 24 well plates in either Insect-XPRESS™ Insect Cell Medium (commercial protein-free media) or ZB Media. Vn-GFP expression was induced one day after seeding with 500 μM copper sulfate. Cells were imaged pre-inductions and then daily after expression was induced. ZB Media cultured cells had higher expression levels of vn-GFP than cells in Insect-XPRESS™ Insect Cell Medium (Lonza). FIG. 21 shows an enlarged picture of Day 4 showing the clearly improved transfection. FIG. 22 shows that ZB Media has lower background fluorescence than Insect Express. Fluorescent intensity (485/528) for vn-GFP stably transfected S2 cells in either ZB Media or Insect Express quadruplicate (same experiment as described for FIG. 20). FIG. 23 illustrates that ZB Media yields comparable transfection efficiency to C1.8 Media for adapted C1.8 cells. C1.8 cells adapted to ZB Media were seeded at 200,000 cell/mL in either C18 or ZB Media. After one day, cells were transiently transfected with B-delta2-HA4CaTTGCamp6Fast using TransIT-X2 transfection reagent. Cells were then imaged 48 hours after transfection cells at 10 second intervals for 2 hours.

ZB Media Profiling.

To study the ability of the ZB media on effective transfection and functional protein expression the insect cell calcium sensor expression vector pAc5-GCaMP6f was transfected into ZB media adapted or into high passage (p68) C1.8 cells. Additionally, the functionality of the calcium sensor cloned into a novel minimal tubulin promoter plasmid pΔTub^{att}-GCaMP6f was preliminary tested using ZB media for transfection and expression.

Methods.

Insect Cell Calcium Sensor DNA Constructs.

The calcium sensor for C1.8 cells transfections was made from plasmid pGP-CMV-GCaMP6f (Addgene #40755). A double digestion of pGP-CMV-GCaMP6f with NotI and BglII created the target fragment ORF (1,352 bp). The sensor was cloned into two different expression vectors:

1) pAc5-STABLE1-blast (Addgene Ac5-STABLE1-neo plasmid #32425 modified version, a gift from Dr. J. D. Sutherland) after removal of the original EGFP module with XbaI and HindIII. Ligation products were screened with PstI for correct orientation of insert.

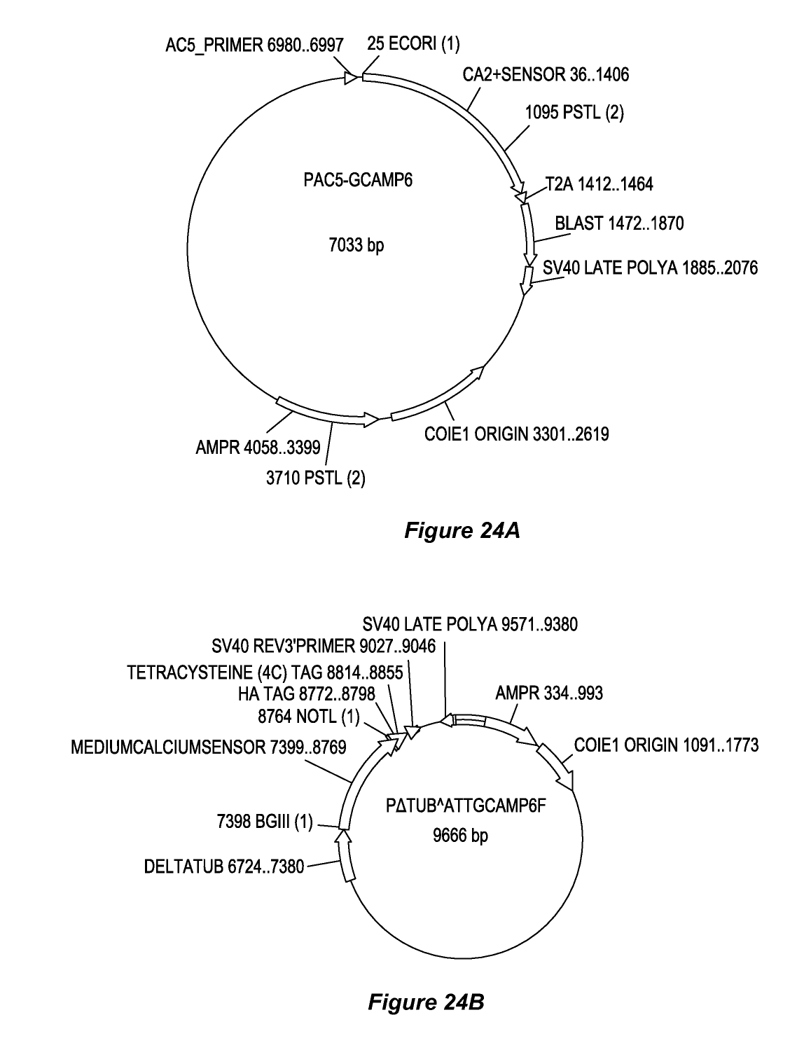

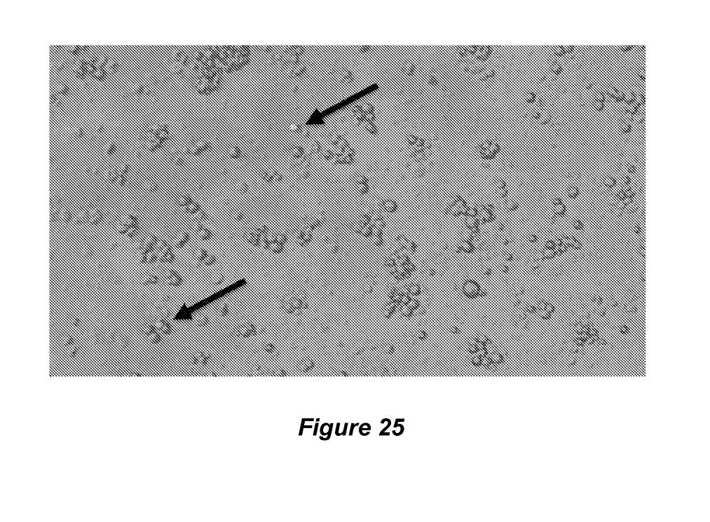

2) pΔTubHA4C^{att} (a gift Dr. D. E. Rincon-Limas (Zhang et al., Mol. Biol. Rep. 40, 5407-5415 (2013))) after digestion with BglII and NotI and directional cloning of the sensor ORF with the insert stop codon (1,352 bp). The final insect cell fast calcium sensor expression constructs: pAc5-GCaMP6f and pΔTub^{att}-GCaMP6f are shown in FIG. 24. High Passage C1.8 cells pΔTub̂att-GCaMP6f Transfection 24 hours post-transfection in Spent ZB Media are shown in FIG. 25.

Cell Culture and Transfections.

Drosophila C1.8 cell line (CME w1 C1.8⁺Melanogaster, dorsal mesothoracic disc, modENCODE line #151) was obtained from the DGRC and maintained in C1.8 media in a humidified incubator at 25° C. in plug seal tissue culture flasks (C1.8 media: M3+2% FCS+5 μg/mL human insulin+2.5% fly extract). To study the ability of the ZB media on effective transfection and functional protein expression the insect cell calcium sensor expression vectors pAc5-GCaMP6f was transfected into C1.8 cells adapted or high passage (p68). The functionality of the calcium sensor cloned into a novel minimal tubulin promoter plasmid pΔTub^{att}-GCaMP6f was preliminary tested using ZB media for transfection and expression.

C1.8 cells were harvested in early exponential phase and seeded into optical-grade 96 well plates the day before transfection at a cell density of 5×10⁵ cells/mL in a final volume of 100 μL per well. The next day the cells were 60-70% confluent at the time of transfection. The calcium sensor plasmids DNA were prepared with EndoFree Plasmid Maxi Kit (Qiagen, 12362). An amount of 0.1 μg of DNA per well was transfected using TransIT-X2 Dynamic Delivery System reagent (Mirus, MIR 6003) following manufacturer's instructions. The Ca²⁺ sensor expression was monitored 24 hours post-transfection by imaging the wells using Evos digital microscope using the GFP channel. Maximum transient expression was found to be 72 hours after transfection.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. An aqueous chemically defined media for supporting long-term growth of Drosophila cell lines, the media comprising ZO media supplemented with insulin, a disaccharide, ascorbic acid, and at least one of glutamine and glutamate, wherein the pH of the media is about 6 to about 8.

2. The media of claim 1 comprising about 95% to about 99% water.

3. The media of claim 1 wherein the insulin is present at a concentration of at least about 1 ng μg/mL.

4. The media of claim 1 wherein the disaccharide is present at a concentration of at least about 10 mM.

5. The media of claim 1 wherein the ascorbic acid is present at a concentration of at least about 40 ng/mL.

6. The media of claim 1 wherein the ascorbic acid is in the form of L-ascorbic acid 2-phosphate sesquimagnesium salt hydrate.

7. The media of claim 1 wherein the glutamine is in the form of a dipeptide.

8. The media of claim 1 wherein at least one of glutamine and glutamate is present at a concentration of at least about 5 μg/mL.

9. The media of claim 1 wherein the pH is about 6.5 to about 7.3.

10. The media of claim 1 further comprising a polyamine compound.

11. The media of claim 10 wherein the polyamine compound is present at a concentration of at least about 0.25 μM.

12. The media of claim 11 wherein the polyamine compound is present at a concentration of about 0.5 μM to about 20 μM.

13. The media of claim 10 wherein the polyamine compound is spermidine, spermine, or putrescine.

14. The media of claim 1 further comprising a BTK inhibitor.

15. The media of claim 10 further comprising a BTK inhibitor.

16. The media of claim 14 wherein the BTK inhibitor is terreic acid or LFM-A13 (α-cyano-β-hydroxy-β-methyl-N-(2,5-dibromophenyl)propenamide).

17. The media of claim 1 wherein the media comprises insulin at a concentration of about 3-10 μg/mL, trehalose at a concentration of about 20-30 mM, L-alanyl-L-glutamine at a concentration of about 10-20 μM, and L-ascorbic acid 2-phosphate sesquimagnesium salt hydrate at a concentration of about 50-150 nM.

18. The media of claim 17 wherein the pH is about 6.7 to about 6.8, the media comprises a polyamine at a concentration of about 0.5 μM to about 20 μM, and the polyamine is spermidine.

19. (canceled)

20. (canceled)

21. An aqueous serum-free chemically defined media for supporting long-term growth of Drosophila cell lines, the media consisting essentially of ZO media supplemented with insulin at a concentration of about 1 ng/mL-10 μg/mL, trehalose at a concentration of about 20-30 mM, L-alanyl-L-glutamine at a concentration of about 1-20 μM, L-ascorbic acid 2-phosphate sesquimagnesium salt hydrate at a concentration of about 40-150 nM, and at least one of glutamine and glutamate at a concentration of about 1 μM to about 20 μM, wherein the pH of the media is about 6 to about 8.

22. A method for screening small molecule compounds comprising:
a) combining Drosophila cells, one or more test compounds, and a Drosophila cell media, wherein the Drosophila cell media is an aqueous chemically defined media comprising ZO media supplemented with insulin, a disaccharide, L-ascorbic acid 2-phosphate, and at least one of glutamine and glutamate, wherein the pH of the media is about 6 to about 8;
b) analyzing the cells after a period of time to score the screen for increases or decreases in the number of cells that are in the presence of the one or more test compounds to obtain compound scores (zᵢ);
c) identifying test compounds that increase the number of Drosophila cells in the media and test compounds that modify the number of Drosophila cells in the media, change the morphology of the cells, or affect the amount of cell signaling as measured by reporter genes or chemical dyes, thereby identifying compounds of interest;
d) querying the Search Tool for Interactions of Chemicals (STITCH) database for all test compounds to determine test compound-protein interaction scores (sᵢ); and
e) optionally identifying species homologs and percent identity matches (qᵢ) for orthologous Drosophila proteins to identified mouse or human protein-drug interactions;
thereby translating screen wide compound proliferation data into biologically relevant protein data by identifying significant protein targets (p-values) from the protein score, Σ(sᵢ·qᵢ·zᵢ²), which follows a chi-squared distribution with degrees of freedom n.

23.-28. (canceled)
